SEQUENCE LISTING (23 sequences)

SEQ ID NO: 1 - DNA, 20 bp, Artificial Sequence
  Synthetic Construct
tccatgacgttcctgacgtt

SEQ ID NO: 2 - DNA, 18 bp, Artificial Sequence
  Synthetic Construct
tctcccagcgtgcgccat

SEQ ID NO: 3 - DNA, 30 bp, Artificial Sequence
  Synthetic Construct
accgatgacgtcgccggtgacggcaccacg

SEQ ID NO: 4 - DNA, 24 bp, Artificial Sequence
  Synthetic Construct
tcgtcgttttgtcgttttgtcgtt

SEQ ID NO: 5 - DNA, 20 bp, Artificial Sequence
  Synthetic Construct
tccatgacgttcctgatgct

SEQ ID NO: 6 - DNA, 22 bp, Artificial Sequence
  Synthetic Construct
caccatgggccagactgttacc

SEQ ID NO: 7 - DNA, 24 bp, Artificial Sequence
  Synthetic Construct
ctactagtcatctagggtcaggag

SEQ ID NO: 8 - DNA, 26 bp, Artificial Sequence
  Synthetic Construct
caccatgaaggcaaacctactggtcc

SEQ ID NO: 9 - DNA, 22 bp, Artificial Sequence
  Synthetic Construct
tcagatgcatattctgcactgc

SEQ ID NO: 10 - DNA, 36 bp, Artificial Sequence
  Synthetic Construct
caccatgaatccaaatcagaaaataataaccattcc

SEQ ID NO: 11 - DNA, 25 bp, Artificial Sequence
  Synthetic Construct
ctacttgtcaatggtgaatggcaac

SEQ ID NO: 12 - DNA, 28 bp, Artificial Sequence
  Synthetic Construct
caccatgaagaccatcattgctttgagc

SEQ ID NO: 13 - DNA, 30 bp, Artificial Sequence
  Synthetic Construct
tcaaatgcaaatgttgcacctaatgttgcc

SEQ ID NO: 14 - DNA, 49 bp, Artificial Sequence
  Synthetic Construct
atataggcgcgccaccatgaatccaaatcaaaagataataacaattggc

SEQ ID NO: 15 - DNA, 40 bp, Artificial Sequence
  Synthetic Construct
atatagcggccgcttatataggcatgaaattgatgttcgc

SEQ ID NO: 16 - DNA, 23 bp, Artificial Sequence
  Synthetic Construct
caccatggagaaaatagtgcttc

SEQ ID NO: 17 - DNA, 26 bp, Artificial Sequence
  Synthetic Construct
ttaaatgcaaattctgcattgtaacg

SEQ ID NO: 18 - DNA, 31 bp, Artificial Sequence
  Synthetic Construct
caccatgaatccaaatcagaagataataacc

SEQ ID NO: 19 - DNA, 22 bp, Artificial Sequence
  Synthetic Construct
ctacttgtcaatggtgaatggc

SEQ ID NO: 20 - DNA, 40 bp, Artificial Sequence
  Synthetic Construct
atataggcgcgccaccatgaagactatcattgctttgagc

SEQ ID NO: 21 - DNA, 43 bp, Artificial Sequence
  Synthetic Construct
atatagcggccgctcaaatgcaaatgttgcacctaatgttgcc

SEQ ID NO: 22 - DNA, 49 bp, Artificial Sequence
  Synthetic Construct
atataggcgcgccaccatgaatccaaatcaaaagataataacgattggc

SEQ ID NO: 23 - DNA, 39 bp, Artificial Sequence
  Synthetic Construct
atatagcggccgcttatataggcatgagattgatgtccg

(19) United States
(12) Patent Application Publication
(10) Pub. No.: US 2011/0262483 A1
(43) Pub. Date: Oct. 27, 2011

(54) METHODS FOR ISOLATING ENVELOPED VIRUS-BASED VLPS FREE OF INFECTIOUS AGENTS

(75) Inventors: Joel R. Haynes, Bozeman, MT (US); Ross Taylor, Bozeman, MT (US)

(73) Assignee: LIGOCYTE PHARMACEUTICALS, INC.

(21) Appl. No.: 13/127,411

(22) PCT Filed: Nov. 3, 2009
(86) PCT No.: PCT/US09/63128

§ 371 Date: Jul. 14, 2011

(30) Foreign Application Priority Data
Nov. 3, 2008 (US) 61110805

(51) Int. Cl. A61K 39/155; A61P 37/04; A61P 31/14; C12N 15/866; A61K 39/12

(52) U.S. Cl. 424/211.1; 435/69.3; 424/204.1

(57) ABSTRACT

Enveloped virus-based virus-like particles (VLPs) that are free of infectious agents and substantially as immunogenic as corresponding VLPs prior to inactivation of infectious agents are described. Improved methods of inactivation infectious agents in preparations of enveloped virus-based VLPs are also described wherein the methods do not adversely affect the immunogenicity of the VLPs.

### FIELD OF THE INVENTION

The present invention relates to the field of isolation of enveloped virus-based virus-like particles (VLPs) free of infectious agents. In preferred examples, the field includes methods of inactivation of infectious agents that do not adversely affect the immunogenicity of the enveloped virus-based VLPs. In certain embodiments, the enveloped virus-based VLPs are produced in insect cell based expression systems.

### BACKGROUND OF THE INVENTION

Production of enveloped virus-based VLPs typically requires expression and assembly of the VLPs in host cell expression systems as assembly often requires accessory factors found in the host cell. Expression of biological components in host cells carries the risk of contamination by infectious agents. For smaller biological components such as proteins and polysaccharides, filter sterilization is commonly used to remove such agents though other methods such as UV inactivation or chemical inactivation are also used (either alone or in conjunction with filter-based methods). For example, photochemical inactivation has been used to inactivate baculovirus when used to express the glycoprotein D gene of Aujeszky's disease virus without any observed reduction in antibody binding to the expressed protein. (See, S. A. Weightman et al., Journal of Virological Methods 81 (1999):179-182). However, for larger biological components such as VLPs, filter sterilization is often not available as the VLPs are often too close in size to infectious agents (e.g., viruses) to be capable of being separated from such infectious agents by filtration. Therefore, other methods need to be used to inactivate infectious agents when preparing VLPs. Such other methods have been tested in inactivation of baculovirus when used to produce porcine parvovirus (PPV—a non-enveloped or capsid virus) VLPs. Rueda et al. compared pasteurization, chemical, and detergent inactivation of baculovirus and found no impact on the immunogenicity of the PPV VLPs. (See, e.g., P. Rueda et al. Vaccine 19 (2001):726-734)

However, it has been unexpectedly found that the typical methods of inactivation of infectious agents when applied to enveloped virus-based VLPs leads to a marked decrease in the immunogenicity of the enveloped virus-based VLP. Thus there is a need for an inactivation method that may be used on enveloped virus-based VLPs that inactivates infectious agents without adversely affecting the immunogenicity of the VLP.

### SUMMARY

Preferred embodiments of the present invention meet this need by providing various methods and compositions as disclosed herein for inactivation of infectious agents that do not adversely affect the immunogenicity of enveloped virus-based VLPs and compositions comprising infectious agent free enveloped virus-based VLP preparations with substantially the same immunogenicity as VLP preparations not subject to inactivation. Such preferred embodiments are based upon the surprising observation that the electromagnetic radiation-based inactivation methods (either alone or with chemicals reactive to the electromagnetic radiation) tested do not result in decreased immunogenicity of the enveloped virus-based VLPs as compared to other purely chemical based inactivation method.

An aspect of the invention includes a method for isolating an enveloped virus-based virus-like particle preparation which is substantially free of infectious agents which comprises (a) separating the enveloped virus-based virus-like particle preparation from host cells used to generate the enveloped virus-based virus-like particle preparation or from a component of the host cells; and (b) applying a sufficient dose of electromagnetic radiation to the enveloped virus-based virus-like particle preparation to inactivate substantially all of infectious agents in the preparation, wherein the enveloped virus-based virus-like particle preparation after step (b) has substantially the same immunogenicity as the enveloped virus-based virus-like particle preparation prior to step (b). In an embodiment, the separating step (a) comprises at least one centrifugation step. In another embodiment that may be combined with the previous embodiment, the separating step (a) may also include at least one filtration step and the at least one filtration step may further be selected from normal flow filtration, ultrafiltration or tangential flow filtration. In another embodiment that may be combined with the previous embodiments, the separating step (a) may also include a chromatographic step and at least one chromatographic step may further be selected from the group consisting of ion-exchange, affinity, hydrophobic interaction, mixed mode, reversed phase, and size exclusion.

In yet another embodiment that may be combined with any of the previous embodiments, the electromagnetic radiation may be selected from the group consisting of visible, x-ray, ultraviolet and gamma radiation and the ultraviolet radiation may further be selected from the group consisting of UV-A, UV-B and UV-C or the ultraviolet radiation may have a wavelength between 320 nm and 400 nm.

In yet another embodiment that may be combined with any of the previous embodiments, the host cells are insect cells or mammalian cells. In yet another embodiment that may be combined with any of the previous embodiments, the host cells are insect cells and the insect cells are infected with a baculovirus expression vector that expresses at least one component of the enveloped virus-based virus-like particle. In yet another embodiment that may be combined with any of the previous embodiments, the host cell is selected from the group consisting of a Bombyx mori host cell, a Spodoptera frugiperda host cell, a Choristoneura fumiferana host cell, a Heliothis virescens host cell, a Heliothis zea host cell, a Helicoverpa zea host cell, a Helicoverpa virescens host cell, a Orgyia pseudotsugata host cell, a Lymantria dispar host cell, a Plutella xylostella host cell, a Malacostoma disstria host cell, a Trichoplusia ni host cell, a Pieris rapae host cell, a Mamestra configurata host cell, a Mamestra brassica host cell, and a Hyalophora cecropia host cell. In yet another embodiment that may be combined with any of the previous embodiments where the host cell is a mammalian cell, the mammalian cell is selected from a MRC-5 cells, a Vero cell, a PER.C6™ cell, a Chinese Hamster Ovary cell, and an HEK293 cell. In yet another embodiment that may be combined with any of the previous embodiments including insect cells as host cells, the dose of electromagnetic radiation is sufficient to inactivate baculovirus in the enveloped virus-based virus-like particle preparation. In yet another embodiment that may be combined with any of the previous embodiments, the host cells are mammalian cells and said mammalian cells are infected with an adenovirus-, an adeno-associated virus, an alphavirus, a herpesvirus-, a poxvirus- or a retrovirus-expression vector that expresses at least one component of the enveloped virus-based virus-like particle. In yet another embodiment that may be combined with any of the previous embodiments including adenovirus-, an adeno-associated virus, an alphavirus, a herpesvirus-, a poxvirus- or a retrovirus-expression vectors, the dose of electromagnetic radiation is sufficient to inactivate the adenovirus, the adeno-associated virus, the alphavirus, the herpesvirus, the poxvirus or the retrovirus, as appropriate, in the enveloped virus-based virus-like particle preparation.

In yet another embodiment that may be combined with any of the previous embodiments, a DNA intercalating compound may be added to the enveloped virus-based virus-like particle preparation prior to the applying step (b) and the DNA intercalating compound may optionally be photoreactive or may be selected from the group consisting of psoralen, isopsoralen, and derivatives and analogs thereof. In yet another embodiment that may be combined with any of the previous embodiments, the electromagnetic radiation may be selected from the group consisting of ultraviolet radiation and visible light.

In yet another embodiment that may be combined with any of the previous embodiments, the method may further comprise: (c) adding an adjuvant to the enveloped virus-based virus-like particle preparation.

In yet another embodiment that may be combined with any of the previous embodiments, the enveloped virus-based virus-like particle is produced in the host cell prior to the separating step (a) by (i) providing one or more expression vectors, together which express a gag polypeptide and a lipid raft-associated polypeptide linked to an antigen, wherein said antigen is not naturally associated with a lipid raft; (ii) introducing said one or more expression vectors into a cell; and (iii) expressing said gag polypeptide and said lipid raft-associated polypeptide linked to an antigen to produce said virus-like particle.

In yet another embodiment that may be combined with any of the previous embodiments, the enveloped virus-based virus-like particle is produced in the host cell prior to the separating step (a) by (i) providing one or more expression vectors, which expresses a respiratory syncytial virus M polypeptide and a respiratory syncytial virus F polypeptide; (ii) introducing said one or more expression vectors into a cell; and (iii) expressing said respiratory syncytial virus M polypeptide and said respiratory syncytial virus F polypeptide to produce said virus-like particle. The preceding embodiment may optionally further express a respiratory syncytial virus G polypeptide from said one or more expression vectors.

In yet another embodiment that may be combined with any of the previous embodiments, the enveloped virus-based virus-like particle is produced in the host cell prior to the separating step (a) by (i) providing one or more expression vectors, which expresses a retroviral gag polypeptide selected from the group consisting of lentivirus and alpha-retrovirus and a respiratory syncytial virus F polypeptide; (ii) introducing said one or more expression vectors into a cell; and (iii) expressing said retroviral gag polypeptide and said respiratory syncytial virus F polypeptide to produce said virus-like particle. The preceding embodiment may optionally further express a respiratory syncytial virus G polypeptide from said one or more expression vectors.

In yet another embodiment that may be combined with any of the previous embodiments, the enveloped virus-based virus-like particle is produced in the host cell prior to the separating step (a) by (i) providing one or more expression vectors, which together express a gag polypeptide and an influenza hemagglutinin polypeptide; (ii) introducing said one or more expression vectors into a cell; and (iii) expressing said gag polypeptide and said influenza hemagglutinin polypeptide to produce said virus-like particle.

In yet another embodiment that may be combined with any of the previous embodiments, wherein the enveloped virus-based virus-like particle is produced in the host cell prior to the separating step (a) by (i) providing one or more expression vectors, which together which express an influenza M1 polypeptide and a hemagglutinin polypeptide; (ii) introducing said one or more expression vectors into a cell; and (iii) expressing said influenza M1 polypeptide and said hemagglutinin polypeptide to produce said virus-like particle. In yet another embodiment that may be combined with any of the previous embodiments including a hemagglutinin polypeptide, the one or more expression vectors may further express a neuraminidase polypeptide. In yet another embodiment that may be combined with any of the previous embodiments including one or more expression vectors, the one or more expression vectors may be a viral vector(s) and the viral vector(s) may be further selected from the group consisting of: a baculovirus, an alphavirus, an adeno-associated virus, an adenovirus, a herpesvirus, a poxvirus and a retrovirus.

In yet another embodiment that may be combined with any of the previous embodiments, at least one component of the enveloped virus-based virus-like particle is expressed in the host cell using a viral vector and optionally the infectious agents comprise the viral vector. In yet another embodiment that may be combined with any of the previous embodiments, the preparation comprises fewer than twenty infectious agents per milliliter; fewer than fifteen infectious agents per milliliter; fewer than ten infectious agents per milliliter; fewer than eight infectious agents per milliliter; fewer than six infectious agents per milliliter; or fewer than five infectious agents per milliliter.

In yet another embodiment that may be combined with any of the previous embodiments, the enveloped virus-based virus-like particle preparation after step (b) has at least fifty percent of the immunogenicity of the enveloped virus-based virus-like particle preparation prior to step (b), at least sixty percent of the immunogenicity of the enveloped virus-based virus-like particle preparation prior to step (b), at least seventy percent of the immunogenicity of the enveloped virus-based virus-like particle preparation prior to step (b), at least eighty percent of the immunogenicity of the enveloped virus-based virus-like particle preparation prior to step (b), at least eighty-five percent of the immunogenicity of the enveloped virus-based virus-like particle preparation prior to step (b), at least ninety percent of the immunogenicity of the enveloped virus-based virus-like particle preparation prior to step (b), or at least ninety-five percent of the immunogenicity of the enveloped virus-based virus-like particle preparation prior to step (b). In yet another embodiment that may be combined with any of the previous embodiments, the enveloped virus-based virus-like particle preparation after being rendered free of infectious agent will have greater immunogenicity than, or increased immunogenicity as compared to, the enveloped virus-based VLP preparation before being rendered free of infectious agent. In yet another embodiment that may be combined with any of the previous embodiments, the enveloped virus-based virus-like particle comprises a hemagglutinin polypeptide and the physical and biochemical integrity of the enveloped virus-based virus-like particle can be measured using a hemagglutination assay which would be a predictor of immunogenicity or the HAI activity in the serum of animals immunized with the enveloped virus-based virus-like particle can be determined as a measure of immunogenicity. In yet another embodiment that may be combined with any of the previous embodiments specifying immunogenicity, the immunogenicity can be directly measured by vaccinating animals with the enveloped virus-based virus-like particle (which can include a respiratory syncytial virus (RSV) polypeptide) and measuring antibody titers by ELISA or virus neutralization assays or by Western blot or measuring T-cell responses by proliferative assays, ELISPOT assays, or cytokine release assays.

Another aspect of the invention includes enveloped virus-based virus-like particle preparations comprising enveloped virus-based virus-like particles that are substantially free of infectious agents wherein the enveloped virus-based virus-like particles have substantially the same immunogenicity as enveloped virus-based virus-like particles that are not substantially free of infectious agents.

In an embodiment, the enveloped virus-based virus-like particles further comprise insect or mammalian glycosylation. In another embodiment which may be combined with the previous embodiment, the insect glycosylation may be further selected from the group consisting of Bombyx mori; Spodoptera frugiperda; Choristoneura fumiferana; Heliothis virescens; Heliothis zea; Helicoverpa zea; Helicoverpa virescens; Orgyia pseudotsugata; Lymantria dispar; Plutella xylostella; Malacostoma disstria; Trichoplusia ni; Pieris rapae; Mamestra configurata; Mamestra brassica; and Hyalophora cecropia. In another embodiment which may be combined with the previous embodiment, the mammalian glycosylation may be further selected from the group consisting of human (including glycosylation as produced by PER.C6™ cells, MRC-5 cells and HEK293 cells), monkey (including glycosylation as produced by Vero cells) and rodent (including glycosylation as produced by Chinese Hamster Ovary cells). In yet another embodiment that may be combined with any of the previous embodiments, the enveloped virus-based virus-like particles further lack one or more defects selected from: covalently linked photochemical agents, UV- or gamma-irradiation induced changes in the tertiary or the quaternary structure of protein subunits, gamma irradiation induced chemical bond cleavage, or UV- or gamma-irradiation induced chemical modifications selected from the group consisting of lipid oxidation, protein crosslinking, amino acid oxidation and amino acid modification. In a preferred embodiment, the enveloped virus-based virus-like particles lack all of the defects. In certain embodiments, the detection of such defects may be as inferred by no decrease in immunogenicity or by application of appropriate technique (e.g., mass spec for confirmation of no defects relating to covalent changes in the polypeptides comprising the virus-like particles and analytical HPLC for confirmation of no defects in tertiary or quaternary structure of the polypeptides comprising the virus-like particles.

In yet another embodiment that may be combined with any of the previous embodiments, the enveloped virus-based virus-like particles comprise a gag polypeptide; and a non-viral lipid raft-associated polypeptide or a lipid raft-associated polypeptide linked to an antigen to form a linkage, wherein said antigen is not naturally associated with a lipid raft and optionally the non-viral lipid raft-associated polypeptide may further be selected from the group consisting of a GPI anchor polypeptide, a myristoylation sequence polypeptide, a palmitoylation sequence polypeptide, a double acetylation sequence polypeptide, a signal transduction polypeptide, and a membrane trafficking polypeptide or from the group consisting of a GPI anchor polypeptide, a myristoylation sequence polypeptide, a palmitoylation sequence polypeptide, a double acetylation sequence polypeptide, a cavelin polypeptide, a flotillin polypeptide, a syntaxin-1 polypeptide, a syntaxin-4 polypeptide, a synapsin I polypeptide, an adducin polypeptide, a VAMP2 polypeptide, a VAMP/synaptobrevin polypeptide, a synaptobrevin II polypeptide, a SNARE polypeptide, a SNAP-25 polypeptide, a SNAP-23 polypeptide, a synaptotagmin I polypeptide, and a synaptotagmin II polypeptide. The viral lipid raft-associated polypeptide may further be selected from the group consisting of: a hemagglutinin polypeptide, a neuraminidase polypeptide, a fusion protein polypeptide, a glycoprotein polypeptide, and an envelope protein polypeptide. In yet another embodiment that may be combined with any of the previous embodiments, the enveloped virus-based virus-like particles further comprise a membrane associated envelope protein polypeptide.

In yet another embodiment that may be combined with any of the previous embodiments including a linkage, the linkage may be further selected from the group consisting of: a covalent bond, an ionic interaction, a hydrogen bond, an ionic bond, a van der Waals force, a metal-ligand interaction, and an antibody-antigen interaction and the covalent bond may optionally be further selected from the group consisting of: a peptide bond, a carbon-oxygen bond, a carbon-sulfur bond, a carbon-nitrogen bond, a carbon-carbon bond, and a disulfide bond. In yet another embodiment that may be combined with any of the previous embodiments including a lipid raft-associated polypeptide, the lipid raft-associated polypeptide is an integral membrane protein. In yet another embodiment that may be combined with any of the previous embodiments including an antigen, the antigen may be further selected from the group consisting of: a protein, a polypeptide, a glycopolypeptide, a lipopolypeptide, a peptide, a polysaccharide, a polysaccharide conjugate, a peptide or non-peptide mimic of a polysaccharide, a small molecule, a lipid, a glycolipid, and a carbohydrate.

In yet another embodiment that may be combined with any of the previous embodiments, the enveloped virus-based virus-like particle further comprises a hemagglutinin polypeptide, a respiratory syncytial virus M polypeptide, a respiratory syncytial virus G polypeptide, and/or a respiratory syncytial virus F polypeptide. In yet another embodiment that may be combined with any of the previous embodiments, the enveloped virus-based virus-like particles comprises a gag polypeptide and a hemagglutinin polypeptide; a retroviral gag polypeptide selected from the group consisting of lentivirus and alpha-retrovirus and a respiratory syncytial virus F polypeptide (and optionally G polypeptide); or an influenza M1 polypeptide and a hemagglutinin polypeptide. In yet another embodiment that may be combined with any of the previous embodiments, the enveloped virus-based virus-like particle further includes a neuraminidase polypeptide.

In yet another embodiment that may be combined with any of the previous embodiments, the enveloped virus-based virus-like particle preparation includes an adjuvant associated with said virus-like particle. In certain embodiments including an adjuvant, the adjuvant may be mixed with the enveloped virus-based virus-like particle during formulation steps. The adjuvant may be located inside or outside or may be integral to the virus-like particle. In yet another embodiment that may be combined with any of the previous embodiments, the adjuvant may be covalently linked to said virus-like particle to form a covalent linkage.

Another aspect of the present invention includes methods for treating or preventing a disease or symptom of the immune system, comprising administering an immunogenic amount of the enveloped virus based virus like particle preparation of any of the preceding embodiments or an enveloped virus based virus like particle preparation isolated using the preceding method and any of its embodiments to a subject. In one embodiment, the subject is human. In another embodiment that can be combined with the previous embodiment, the administering induces a protective immunization response in the subject. In yet another embodiment that may be combined with any of the previous embodiments, the administering may further be selected from the group consisting of subcutaneous delivery, intradermal delivery, subdermal delivery, intramuscularly delivery, peroral delivery, oral delivery, intranasal delivery, buccal delivery, sublingual delivery, intraperitoneal delivery, intravaginal delivery, anal delivery and intracranial delivery.

Another aspect of the present invention includes pharmaceutical compositions that comprising an immunogenic amount of the enveloped virus based virus like particle preparation of any of the preceding embodiments or an enveloped virus based virus like particle preparation isolated using the preceding method and any of its embodiments.

The foregoing aspects and embodiments thereof may further be combined with any of the embodiments disclosed in the specification. Additional aspects of the invention may be found throughout the specification which may be included with any of foregoing embodiments and/or the additional embodiments disclosed in the specification

### SUMMARY OF THE FIGURES

FIG. 1 shows the analysis of chimeric VLPs containing HA and NA. Supernatants of recombinant baculovirus-expressing cells were cleared of cellular debris and PR/8 H1N1 VLPs were centrifuged at 100,000×g through a sucrose cushion, resuspended and centrifuged again on a discontinuous sucrose density gradient. (A) PR/8 H1N1 VLP hemagglutination activity across the gradient. (B) SDS-PAGE analysis of PR/8 H1N1 VLPs showing co-migrating Gag and HA. (C) H1N1-specific Western blot of gradient fractions 4-16 using an antibody specific for A/Russia/77 (H1N1). (D) Electron micrograph of negative stained sample from peak gradient fraction. (E) SDS-PAGE of purified PR/8 H1N1 VLPs in which the HA gene was extended at its amino terminus with irrelevant sequences to increase the molecular weight and reduce the electrophoretic mobility of the HA product. (F) SDS-PAGE of purified VLPs representing A/Solomon Islands/3/2006 (H1N1) showing the ratio of Gag-to-HA.

FIG. 2 shows the examination of hemagglutination and neuraminidase activity in H5N1 VLPs centrifuged on discontinuous sucrose gradients. H5N1 VLPs were prepared as shown for H1N1 VLPs in FIG. 1 but sucrose gradients fractions were assayed for both hemagglutination and neuraminidase activity. NA activity was measured using the fluorescent substrate 2′-(4-methylumbelliferyl)-α-D-N-acetylneuraminic acid. (A) shows activities for Vietnam H5N1 VLPs. (B) shows activities for Indonesia H5N1 VLPs.

FIG. 3 shows VLP immunogenicity and challenge protection. Three groups of 16 mice received primary and booster immunizations with chimeric H1N1 VLPs (representing A/PR/8/34) via i.m. or i.p. immunization. All VLP formulations contained approximately 0.7 μg HA per dose. Animals were challenged with 10 LD50 of A/PR/8/34 (H1N1) four weeks following the boost. (A) HAI activity specific for A/PR/8/34 (H1N1) two weeks post-boost. (B) Quantification of A/PR/8/34-specific IgG1 and IgG2A responses two weeks post-boost. (C) Challenge protection as shown by weight loss following H1N1 challenge.

FIG. 4 shows that VLPs and live virus perform similarly in HAI assays. Sera from mice immunized with PR/8 (H1N1) VLPs were tested for HAI activity using either 4 HA units of live PR/8 (H1N1) virus or 4 HA units of the corresponding VLP. Sera from mice immunized with HK/68 (H3N2) VLPs were similarly tested against HK/68 virus and VLPs. Data showed similar performance between virus and VLPs in the HAI assay.

FIG. 5 shows post-boost immune responses in VLP-immunized ferrets. Ferrets received primary and booster immunizations on days 0 and 28, respectively, with H1N1, H5N1, and naked VLPs. H1N1 and H5N1 VLP doses contained approximately 5 μg HA per dose. Day 28 and 42 serum samples were analyzed for (A) A/PR/8/34 (H1N1)-specific HAI activity and (B) A/Vietnam/1203/04 (H5N1)-specific microneutralization activity.

FIG. 6 shows post-challenge weight loss and survival in ferrets immunized with H1N1, H5N1 and naked VLPs. Two weeks after receipt of the booster immunization, VLP-vaccinated ferrets were challenged with 1×106 TCID50 of A/Vietnam/1203/04 (H5N1). (A) Mean weight loss data for all groups (survival data is shown in the graph legend). (B) Individual weight loss data for H1N1-vaccinated animals in Group B.

FIG. 7 shows post-challenge nasal wash virus titers in ferrets immunized with H1N1, H5N1, and naked VLPs. Two weeks after receipt of the booster immunization, VLP-vaccinated ferrets were challenged with 1×106 TCID50 of A/Vietnam/1203/04 (H5N1). (A) Nasal wash virus titers on day 3 post-challenge (day 45). (B) Nasal wash virus titers on day 5 post-challenge (day 47).

### DETAILED DESCRIPTION OF THE PREFERRED EMBODIMENTS

Preferred embodiments of the present invention include, without limitation, enveloped virus-based VLPs preparations that have been subject to a method that inactivates infectious agents which allows the VLPs to retain substantially the same immunogenicity as VLPs that have not been subject to such inactivation method; such methods of inactivating infectious agents in enveloped virus-based VLPs preparations; methods of further processing such preparations into vaccine compositions and methods of using such vaccine compositions.

While not wanting to be limited to theory, certain preferred embodiments of the invention are based upon the surprising discovery that, unlike other non-enveloped virus-based VLPs such as PPV VLPs, enveloped virus-based VLPs have been discovered to be sensitive to the standard inactivation methods employed to inactivate infectious agents as set forth in the examples below. However, equally surprising, electromagnetic based inactivation systems, such as UV-A+photochemical agent, UV-C, and gamma irradiation, are effective at inactivating contaminating enveloped viruses while maintaining the immunogenicity of the envelope virus-based VLPs thus making such inactivation methods ideal for preparing enveloped virus-based VLPs for use in vaccines.

A preferred method of generating the enveloped virus-based VLPs is by expression in insect cells, preferably including coexpression of polypeptide antigens. Even more preferably, the VLP is generated using a gag polypeptide, because of the significant yields of gag VLPs that can be obtained from a variety of retroviruses in the baculovirus expression system (23, 24, 46, 49, 52-58). Gag polypeptides inherently include C-terminal extensions in the natural retroviral assembly process in that functional gag proteins naturally have large C-terminal extensions containing retroviral protease, reverse transcriptase, and integrase activity due to ribosomal frameshifting. Production of functional gag proteins with artificial extensions has been accomplished for both rous sarcoma virus gag (59) and MLV gag (60). This flexibility in manipulation of the gag C-terminus provides an important site for inclusion of other polypeptides such as other antigens and immunostimulatory protein sequences. Another preferred method of generating the enveloped virus-based VLPs is by coexpression of the influenza HA and M1 proteins (and optionally influenza NA). Coexpression of these two proteins in insect cells has been shown to be an effective method of generating enveloped virus-based VLPs (111-112).

Preferred examples of enveloped virus-based VLPs include VLPs that comprise: (i) a gag polypeptide and a lipid raft-associated polypeptide linked to an antigen, (ii) a respiratory syncytial virus M polypeptide and a respiratory syncytial virus F polypeptide (and optionally a respiratory syncytial virus G polypeptide), (iii) a retroviral gag polypeptide selected from the group consisting of lentivirus and alpha-retrovirus and a respiratory syncytial virus F polypeptide, (iv) a gag polypeptide and an influenza hemagglutinin polypeptide (and optionally a neuraminidase polypeptide); and (v) an influenza M1 polypeptide and a hemagglutinin polypeptide (and optionally a neuraminidase polypeptide.

The production of chimeric VLPs containing a core particle from one virus and surface antigens from another is called pseudotyping. Gag polypeptides have been efficiently pseudotyped with influenza HA and NA, presumably since these proteins are concentrated within lipid raft domains (61, 62) while myristolated gag proteins also concentrate at the inner surface of lipid raft domains during the budding process (63).

The embodiments of the present invention described herein are compatible with VLP platforms that include lipid-raft associated polypeptides linked to an antigen which is not naturally associated with a lipid raft as a basis for formation of chimeric VLPs.

The practice of the disclosed methods and protocols will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

### DEFINITIONS

An “enveloped virus-based VLP” as used here refers to virus-like particles that are formed using one or more components derived from an enveloped virus. Preferred examples include, without limitation, VLPs generated using gag polypeptides, VLPs generated using influenza M1 polypeptides and/or hemagglutinin polypeptides (and optionally neuraminidase polypeptides), VLPs generated using the group consisting of lentivirus and alpha-retrovirus gag polypeptides and a respiratory syncytial virus (RSV) F polypeptide (and optionally G polypeptide), and VLPs generated using respiratory syncytial virus (RSV) M and/or F polypeptides (and optionally G polypeptide).

Additional examples include: filoviruses such as Ebola virus and Marburg virus may be used to form enveloped virus based VLPs (e.g., coexpression of virus GP and VP40 from filoviruses in cells will generate VLPs owing to the association of these two viral proteins in lipid rafts (see U.S. Pat. Publ. 20060099225)); coronaviruses such as SARS (e.g., E and M proteins are sufficient for coronavirus VLP formation (see Fischer et al., J. Virol. (1998) 72:7885-7894 and Vennema et al. EMBO J. (1996) 15:2020-2028)); paramyxoviridae viruses such as respiratory syncytial virus (RSV) (e.g., expression of the M protein of RSV will generate VLPs (See, e.g., U.S. Pat. Publ. 20080233150)); and flaviviridae such as West Nile Virus (e.g., expressing a construct comprising the prM and E genes of a West Nile Virus in baculovirus expression system will generate VLPs (See, e.g., U.S. Pat. Publ. 20080233150)).

As used herein, “free of infectious agent” refers to the absence of active agents that are capable of infection. Such a sample may contain agents that are inactive and are not capable of infection. By way of example, a sample containing baculovirus that has been treated such that the baculovirus is no longer capable of infection is free of infectious agent even though the sample still contains inactivated baculovirus. Furthermore, a sample need not be absolutely free of active agent capable of infection, but rather, the sample need only be sufficiently free of active agent so that the sample may be used for its intended purpose as a human or animal vaccine, as applicable, (i.e., it meets any United States federal regulations governing the acceptable levels of infectious agent within a human or animal vaccine, as applicable). In certain embodiments, the enveloped virus-based VLP preparation after being rendered free of infectious agent will have the infectious units per dose or infectious units per ml reduced by at least 10-fold, by at least 100-fold, by at least 1000-fold, or by at least 10,000-fold of the enveloped virus-based VLP preparation before being rendered free of infectious agent. By way of example, infectious agents can include the vector(s) used to express the polypeptide(s) comprising the VLPs in one or more host cells, as well as external bacterial, fungal or viral contaminants and even endogenous pathogens (e.g., derived from source material or host cell such as reactivated retroviral or retrotransposable elements typically silent in the host genome).

As used herein, “substantially the same immunogenicity” refers to the immunogenicity of the enveloped virus-based VLP preparation after the preparation has been rendered free of infectious agent as compared to the preparation before it has been rendered free of infectious agent. In certain embodiments, the enveloped virus-based VLP preparation after being rendered free of infectious agent will have at least fifty percent, at least sixty percent, at least seventy percent, at least eighty percent, at least eighty-five percent, at least ninety percent, or at least ninety-five percent of the immunogenicity of the enveloped virus-based VLP preparation before being rendered free of infectious agent. In certain embodiments, the enveloped virus-based VLP preparation after being rendered free of infectious agent will have greater immunogenicity than, or increased immunogenicity as compared to, the enveloped virus-based VLP preparation before being rendered free of infectious agent. A preferred measure of immunogenicity is titer of antibody to VLP compositions produced after inoculation. For influenza vaccines, a preferred measure of immunogenicity is the HAI activity in accordance with the examples below. In yet another embodiment that may be combined with any of the previous embodiments, the enveloped virus-based virus-like particle comprises a hemagglutinin polypeptide and the physical and biochemical integrity of the enveloped virus-based virus-like particle can be measured using a hemagglutination assay which would be a predictor of immunogenicity or the HAI activity in the serum of animals immunized with the enveloped virus-based virus-like particle can be determined as a measure of immunogenicity. In yet another embodiment that may be combined with any of the previous embodiments specifying immunogenicity, the immunogenicity can be directly measured by vaccinating animals with the enveloped virus-based virus-like particle (which can include a respiratory syncytial virus (RSV) polypeptide) and measuring antibody titers by ELISA or virus neutralization assays or by Western blot or measuring T-cell responses by proliferative assays, ELISPOT assays, or cytokine release assays.

“Insect glycosylation” refers to glycosylation patterns generated by insects and by insect cell-based expression systems. Such glycosylation patterns can include both naturally produced glycosylation as well as glycosylation patterns produced by insect cells that have been modified to include mammalian glycosylation enzymes, so long as such modified insect cells only produce “mammalian-like” glycosylation rather than the glycosylation pattern that would be naturally produced by a mammal or a mammalian cell based expression system. Preferred examples of insect glycosylation patterns include insect cells which are compatible with the baculovirus and related-viral expression systems such as Bombyx mori; Spodoptera frugiperda; Choristoneura fumiferana; Heliothis virescens; Heliothis zea; Helicoverpa zea; Helicoverpa virescens; Orgyia pseudotsugata; Lymantria dispar; Plutella xylostella; Malacostoma disstria; Trichoplusia ni; Pieris rapae; Mamestra configurata; Mamestra brassica; and Hyalophora cecropia.

“Mammalian glycosylation” refers to glycosylation patterns generated by mammals and by mammalian cell-based expression systems. Such glycosylation patterns can include both naturally produced glycosylation as well as glycosylation patterns produced by mammalian cells that have been modified to include glycosylation enzymes not found or not typically expressed in such cell, so long as such modified mammalian cells only produce mammalian or unnatural glycosylation rather than the glycosylation pattern that would be naturally produced by a non-mammal or a non-mammalian cell based expression system. Preferred examples of insect glycosylation patterns include mammalian cells which are compatible with known viral expression systems such as: human cells (including glycosylation as produced by PER.C6™ cells, MRC-5 cells and HEK293 cells), monkey cells (including glycosylation as produced by Vero cells) and rodent cells (including glycosylation as produced by Chinese Hamster Ovary cells).

The “Gag polypeptide” as used herein is the retrovirus-derived structural polypeptide that is responsible for formation of the virus-like particles described herein. The gag polypeptide is a preferred means of forming enveloped virus based VLPs. In some embodiments, the gag polypeptide may be purposely mutated in order to affect certain characteristics such as the propensity to package RNA or the efficiency of particle formation and budding. The genome of retroviruses codes for three major gene products: the gag gene coding for structural proteins, the pol gene coding for reverse transcriptase and associated proteolytic polypeptides, nuclease and integrase associated functions, and env whose encoded glycoprotein membrane proteins are detected on the surface of infected cells and also on the surface of mature released virus particles. The gag genes of all retroviruses have an overall structural similarity and within each group of retroviruses are conserved at the amino acid level. The gag gene gives rise to the core proteins excluding the reverse transcriptase.

For MLV the Gag precursor polyprotein is Pr65^{Gag} and is cleaved into four proteins whose order on the precursor is NH₂-p15-pp12-p30-p10-COOH. These cleavages are mediated by a viral protease and may occur before or after viral release depending upon the virus. The MLV Gag protein exists in a glycosylated and a non-glycosylated form. The glycosylated forms are cleaved from gPr80^{Gag} which is synthesized from a different inframe initiation codon located upstream from the AUG codon for the non-glycosylated Pr65^{Gag}. Deletion mutants of MLV that do not synthesize the glycosylated Gag are still infectious and the non-glycosylated Gag can still form virus-like particles, thus raising the question over the importance of the glycosylation events. The post translational cleavage of the HIV-1 Gag precursor of pr55^{Gag} by the virus coded protease yields the N-myristoylated and internally phosphorylated p17 matrix protein (p17MA), the phosphorylated p24 capsid protein (p24CA), and the nucleocapsid protein p15 (p15NC), which is further cleaved into p9 and p6.

Structurally, the prototypical Gag polyprotein is divided into three main proteins that always occur in the same order in retroviral gag genes: the matrix protein (MA) (not to be confused with influenza matrix protein M1, which shares the name matrix but is a distinct protein from MA), the capsid protein (CA), and the nucleocapsid protein (NC). Processing of the Gag polyprotein into the mature proteins is catalyzed by the retroviral encoded protease and occurs as the newly budded viral particles mature. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. The form of the Gag protein that mediates assembly is the polyprotein. Thus, the assembly domains need not lie neatly within any of the cleavage products that form later. The Gag polypeptide as included herein therefore includes the important functional elements for formation and release of the VLPs. The state of the art is quite advanced regarding these important functional elements. See, e.g., Hansen et al. J. Virol 64, 5306-5316, 1990; Will et al., AIDS 5, 639-654, 1991; Wang et al. J. Virol. 72, 7950-7959, 1998; McDonnell et al., J. Mol. Biol. 279, 921-928, 1998; Schultz and Rein, J. Virol. 63, 2370-2372, 1989; Accola et al., J. Virol. 72, 2072-2078, 1998; Borsetti et al., J. Virol., 72, 9313-9317, 1998; Bowzard et al., J. Virol. 72, 9034-9044, 1998; Krishna et al., J. Virol. 72, 564-577, 1998; Wills et al., J. Virol. 68, 6605-6618, 1994; Xiang et al., J. Virol. 70, 5695-5700, 1996; Garnier et al., J. Virol. 73, 2309-2320, 1999.

As used in certain VLPs of the present invention, the gag polypeptide shall at a minimum include the functional elements for formation of the VLP. The gag polypeptide may optionally include one or more additional polypeptides that may be generated by splicing the coding sequence for the one or more additional polypeptides into the gag polypeptide coding sequence. A preferred site for insertion of additional polypeptides into the gag polypeptide is the C-terminus.

Preferred retroviral sources for Gag polypeptides include murine leukemia virus, human immunodeficiency virus, Alpharetroviruses (such as the avian leucosis virus or the Rous sarcoma virus), Betaretroviruses (such as mouse mammary tumor virus, Jaagsiekte sheep retrovirus and Mason-Phizer monkey virus), Gammaretroviruses (such as murine leukemia virus, feline leukemia virus, reticuloendotheliosis virus and gibbon ape leukemia virus), Deltaretroviruses (such as human T-lymphotrophic virus and bovine leukemia virus), Epsilonretroviruses (such as walleye dermal sarcoma virus), or Lentiviruses (human immunodeficiency virus type 1, HIV-2, simian immunodeficiency virus, feline immunodeficiency virus, equine infectious anemia virus, and caprine arthritis encephalitis virus).

The “lipid raft” as used herein refers to the cell membrane microdomain in which the gag polypeptide concentrates during the viral particle assembly process.

A “lipid raft-associated polypeptide” as used herein refers to any polypeptide that is directly or indirectly associated with a lipid raft. The particular lipid raft-associated polypeptide used in the invention will depend on the desired use of the chimeric virus-like particle.

The lipid raft-associated polypeptide can be an integral membrane protein, a protein directly associated with the lipid raft via a protein modification which causes association with the membrane, or a polypeptide with an indirect association with the lipid raft via a lipid raft-associated polypeptide.

Many proteins with lipid anchors associate with lipid rafts. Lipid anchors that couple polypeptides to lipid rafts include GPI anchors, myristoylation, palmitoylation, and double acetylation.

Many different types of polypeptides are associated with lipid rafts. Lipid rafts function as platforms for numerous biological activities including signal transduction, membrane trafficking, viral entry, viral assembly, and budding of assembled particles and are therefore associated with the various polypeptides involved in these processes.

The various types of polypeptides involved in signaling cascades are associated with lipid rafts that function as signaling platforms. One type of lipid raft which functions as signaling platform is called a caveolae. It is a flask shaped invagination of the plasma-membrane which contains polypeptides from the caveolin family (e.g., caveolin and/or flottillin).

Membrane trafficking polypeptides are associated with lipid rafts which function as membrane trafficking platforms. Examples include the proteins involved in endocytosis and excocytosis, such as syntaxin-1, syntaxin-4, synapsin I, adducin, VAMP2, VAMP/synaptobrevin, synaptobrevin II, SNARE proteins, SNAP-25, SNAP-23, synaptotagmin I, synaptotagmin II, and the like.

Viral receptors, receptor-coreceptor complexes, any other components which help modulate the entry process are associated with lipid rafts which function as specialized membrane trafficking platforms for viral entry. Examples of lipid raft-associated viral receptors include the decay accelerating factor (DAF or CD55), a GPI-anchored membrane glycoprotein that is a receptor for many enteroviruses; the receptor for group A rotaviruses, a complex containing multiple components including gangliosides, Hsc70 protein, alpha2-beta1 and alpha5-beta2 integrins; glycoproteins of several enveloped viruses like HIV, MLV, measles, and Ebola; and polypeptides involved in HIV entry like CD5, CCR5, and nef. See Chazal and Gerlier, 2003, Virus Entry, Assembly, Budding, and Membrane Rafts, Microbiol. & Mol. Bio. Rev. 67(2):226-237.

Polypeptides involved in viral particle assembly are associated with lipid rafts functioning as viral assembly platforms. Examples of such polypeptides include the HA and NA influenza envelope glycoproteins, the H and mature F1-F2 fusion proteins from measles, and the gp160, gp41, and Pr55gag from HIV. See Chazal and Gerlier, 2003, Virus Entry, Assembly, Budding, and Membrane Rafts, Microbiol. And Mol. Bio. Rev. 67(2):226-237.

Polypeptides involved in budding of assembled virus are associated with lipid rafts that function as viral budding platforms. There is data suggesting that HIV-1 budding from the host cell occurs in membrane rafts. See Chazal and Gerlier, 2003, Virus Entry, Assembly, Budding, and Membrane Rafts, Microbiol. And Mol. Bio. Rev. 67(2):226-237. General information about polypeptides involved in viral budding can be found in Fields Virology (4th ed.) 2001.

Preferred lipid-raft associated polypeptides include viral polypeptides such as hemagglutinin polypeptide, neuraminidase polypeptide, fusion protein polypeptide, glycoprotein polypeptide, and envelope protein polypeptide. Each of these polypeptide can be from any type of virus; however, certain embodiments include envelope protein from HIV-1 virus, fusion protein from respiratory syncytial virus or measles virus, glycoprotein from respiratory syncytial virus, herpes simplex virus, or Ebola virus, and hemagglutinin protein from measles virus.

Preferred non-viral pathogen lipid-raft associated polypeptides may be obtained from pathogenic protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like. Such non-viral lipid-raft associated polypeptides may be used without being liked to an antigen not naturally associated with a lipid-raft as the lipid raft-associated polypeptide itself will act as the antigen.

The “influenza M1 polypeptide” as used herein is derived from the influenza virus protein that mediates formation of the viral coat inside the viral envelope. The M1 protein drives viral budding and is also the dominant protein component of the viral particle, where it forms an intermediate layer between the viral envelope and integral membrane proteins and the genomic ribonucleoproteins. The M1 polypeptide is also thought to bind to the viral RNA in a non-specific manner that is dependent upon a binding motif (RKLKR) in the M1 polypeptide that is rich in positively charged amino acids.

A preferred example of a viral lipid-raft associated polypeptide is a hemagglutinin polypeptide. The “hemagglutinin polypeptide” as used herein is derived from the influenza virus protein that mediates binding of the virus to the cell to be infected. Hemagglutinin polypeptides may also be derived from the comparable measles virus protein. The protein is an antigenic glycoprotein found anchored to the surface of influenza viruses by a single membrane spanning domain. At least sixteen subtypes of the influenza hemagglutinin have been identified labeled H1 through H16. H1, H2, and H3, are found in human influenza viruses. Highly pathogenic avian flu viruses with H5 or H7 hemagglutinins have been found to infect humans at a low rate. It has been reported that single amino acid changes in the avian virus strain's type H5 hemagglutinin have been found in human patients that alters the receptor specificity to allow the H5 hemagglutinin to significantly alter receptor specificity of avian H5N1 viruses, providing them with an ability to bind to human receptors (109 and 110). This finding explains how an H5N1 virus that normally does not infect humans can mutate and become able to efficiently infect human cells.

Hemagglutinin is a homotrimeric integral membrane polypeptide. The membrane spanning domain naturally associates with the raft-lipid domains, which allows it to associate with the gag polypeptides for incorporation into VLPs. It is shaped like a cylinder, and is approximately 135 Å long. The three identical monomers that constitute HA form a central coiled-coil and a spherical head that contains the sialic acid binding sites, which is exposed on the surface of the VLPs. HA monomers are synthesized as a single polypeptide precursor that is glycosylated and cleaved into two smaller polypeptides: the HA1 and HA2 subunits. The HA2 subunits form the trimeric coiled-coil that is anchored to the membrane and the HA1 subunits form the spherical head.

As used in certain VLPs of the present invention as a lipid-raft associated polypeptide, the hemagglutinin polypeptide shall at a minimum include the membrane anchor domain. The hemagglutinin polypeptide may be derived from any influenza virus type, subtype, strain or substrain, preferable from the H1, H2, H3, H5, H7, and H9 hemagglutinins. In addition, the hemagglutinin polypeptide may be a chimera of different influenza hemagglutinins. The hemagglutinin polypeptide preferably includes one or more additional antigens not naturally associated with a lipid raft that may be generated by splicing the coding sequence for the one or more additional polypeptides into the hemagglutinin polypeptide coding sequence. A preferred site for insertion of additional polypeptides into the hemagglutinin polypeptide is the N-terminus.

As used in certain VLPs of the present invention as a antigen, the hemagglutinin polypeptide shall at a minimum include the membrane anchor domain and at least one epitope from hemagglutinin. The hemagglutinin polypeptide may be derived from any influenza virus type, subtype, strain or substrain, preferably from the H1, H2, H3, H5, H7 and H9 hemagglutinins. In addition, the hemagglutinin polypeptide may be a chimera of different influenza hemagglutinins. The hemagglutinin polypeptide may optionally include one or more additional polypeptides that may be generated by splicing the coding sequence for the one or more additional polypeptides into the hemagglutinin polypeptide coding sequence. A preferred site for insertion of additional polypeptides into the hemagglutinin polypeptide is the N-terminus.

Another preferred example of a viral lipid-raft associated polypeptide is a neuraminidase polypeptide. The “neuraminidase polypeptide” as used herein is derived from the influenza virus protein that mediates release of the influenza virus from the cell by cleavage of terminal sialic acid residues from glycoproteins. The neuraminidase glycoprotein is expressed on the viral surface. The neuraminidase proteins are tetrameric and share a common structure consisting of a globular head with a beta-pinwheel structure, a thin stalk region, and a small hydrophobic region that anchors the protein in the virus membrane by a single membrane spanning domain. The active site for sialic acid residue cleavage includes a pocket on the surface of each subunit formed by fifteen charged amino acids, which are conserved in all influenza A viruses. At least nine subtypes of the influenza neuraminidase have been identified labeled N1 through N9.

As used in certain VLPs of the present invention, the neuraminidase polypeptide shall at a minimum include the membrane anchor domain. The state of the art regarding functional regions is quite high. See, e.g., Varghese et al., Nature 303, 35-40, 1983; Colman et al., Nature 303, 41-44, 1983; Lentz et al., Biochem, 26, 5321-5385, 1987; Webster et al., Virol. 135, 30-42, 1984. The neuraminidase polypeptide may be derived from any influenza virus type, subtype strain or substrain, preferably from the N1 and N2 neuraminidases. In addition, the neuraminidase polypeptide may be a chimera of different influenza neuraminidase. The neuraminidase polypeptide preferably includes one or more additional antigens that are not naturally associated with a lipid raft that may be generated by splicing the coding sequence for the one or more additional polypeptides into the hemagglutinin polypeptide. A preferred site for insertion of additional polypeptides into the neuraminidase polypeptide coding sequence is the C-terminus.

As used in certain VLPs of the present invention as an antigen, the neuraminidase polypeptide shall at a minimum include the membrane anchor domain and at least the sialic acid residue cleavage activity. The state of the art regarding functional regions is quite high. See, e.g., Varghese et al., Nature 303, 35-40, 1983; Colman et al., Nature 303, 41-44, 1983; Lentz et al., Biochem, 26, 5321-5385, 1987; Webster et al., Virol. 135, 30-42, 1984. The neuraminidase polypeptide may be derived from any influenza virus type, subtype strain or substrain, preferable from the N1 and N2 neuraminidases. In addition, the neuraminidase polypeptide may be a chimera of different influenza neuraminidase. The neuraminidase polypeptide may optionally include one or more additional polypeptides that may be generated by splicing the coding sequence for the one or more additional polypeptides into the neuraminidase polypeptide coding sequence. A preferred site for insertion of additional polypeptides into the neuraminidase polypeptide is the C-terminus.

Another preferred example of a lipid raft associated peptide is an insect derived adhesion protein termed fasciclin I (FasI). The “fasciclin I polypeptide” as used herein is derived from the insect protein that is involved in embryonic development. This non-viral protein can be expressed in an insect cell baculovirus expression system leading to lipid raft association of FasI (J. Virol. 77, 6265-6273, 2003). It therefore follows that attachment of a heterologous antigen to a fasciclin I polypeptide will lead to incorporation of the chimeric molecule into VLPs when co-expressed with gag. As used in the VLPs of the present invention, the fasciclin I polypeptide shall at a minimum include the membrane anchor domain.

Another preferred example of a lipid raft associated peptide is a viral derived attachment protein from RSV named the G glycoprotein. The “G glycopolypeptide” as used herein is derived from the RSV G glycoprotein. Recent data has demonstrated that lipid raft domains are important for RSV particle budding as they are for influenza virus (Virol 327, 175-185, 2004; Arch. Virol. 149, 199-210, 2004; Virol. 300, 244-254, 2002). The G glycoprotein from RSV is a 32.5 kd integral membrane protein that serves as a viral attachment protein as well as a protective antigen for RSV infection. As with the hemagglutinin from influenza virus, its antigenicity may enhance the antigenicity of any non-lipid raft antigens attached to it. Since RSV does not naturally express a protein with neuraminidase activity, it is likely that VLPs composed of gag and RSV G will not require the presence of NA for efficient production and release. Therefore, development of an expression vector encoding gag (such as an alpha-retrovirus gag) and a G glycopolypeptide will result in the production of VLPs containing the G glycopolypeptide integrated into the membrane. Any modifications to the G glycopolypeptide in the way of non-lipid raft foreign antigen attachment will result in chimeric VLPs capable of inducing significant immune responses to the foreign antigen.

The terms “enveloped virus-based virus-like particle” and “VLP” are used interchangeably throughout except where VLP by its context is referring to a virus-like particle that is not based upon an enveloped based virus or is based upon a particular component of certain enveloped-based viruses as disclosed herein.

Antigens

Certain aspects of the present invention include additional antigens associated with the enveloped virus-based VLP preparations. Such additional antigens may be included in the same composition and may further be covalently or non-covalently associated with the VLPs. In preferred embodiments, gag polypeptides, influenza M1 polypeptides, hemagglutinin polypeptides, neuraminidase polypeptides and/or other lipid raft-associated polypeptides are a readily adaptable platform for forming enveloped virus-based VLPs containing antigens which may not be naturally associated with a lipid raft. This section describes preferred antigens for use with the disclosed VLPs.

### Linkage Between Antigen and Livid Raft-Associated Polypeptide

As a means for forming VLPs containing antigens not naturally associated with a lipid raft, or antigen not naturally associated with the cell membrane, a linkage may be formed between a gag polypeptide, an influenza M1 polypeptide, a hemagglutinin polypeptide, a neuraminidase polypeptide and/or another lipid raft-associated polypeptide and the antigen. The lipid-raft associated polypeptide may be linked to a single antigen or to multiple antigens to increase immunogenicity of the VLP, to confer immunogenicity to various pathogens, or to confer immunogenicity to various strains of a particular pathogen.

The linkage between the antigen and a lipid raft-associated polypeptide can be any type of linkage sufficient to result in the antigen being incorporated into the VLP. The bond can be a covalent bond, an ionic interaction, a hydrogen bond, an ionic bond, a van der Waals force, a metal-ligand interaction, or an antibody-antigen interaction. In preferred embodiments, the linkage is a covalent bond, such as a peptide bond, carbon-oxygen bond, a carbon-sulfur bond, a carbon-nitrogen bond, a carbon-carbon bond, or a disulfide bond.

The antigen may be produced recombinantly with an existing linkage to the lipid-raft associated polypeptide or it may be produced as an isolated substance and then linked at a later time to the lipid-raft associated polypeptide.

Antigen Types

The antigens as used herein can be any substance capable of eliciting an immune response and which does not naturally associate with a lipid raft. Antigens include, but are not limited to, proteins, polypeptides (including active proteins and individual polypeptide epitopes within proteins), glycopolypeptides, lipopolypeptides, peptides, polysaccharides, polysaccharide conjugates, peptide and non-peptide mimics of polysaccharides and other molecules, small molecules, lipids, glycolipids, and carbohydrates. If the antigen does not naturally associate either directly or indirectly with a lipid raft, it would not be expected to be incorporated into a VLP without linkage to a lipid raft-associated polypeptide. The antigen can be any antigen implicated in a disease or disorder, e.g., microbial antigens (e.g., viral antigens, bacterial antigens, fungal antigens, protozoan antigens, helminth antigens, yeast antigens, etc.), tumor antigens, allergens and the like.

Sources for Antigens

The antigens described herein may be synthesized chemically or enzymatically, produced recombinantly, isolated from a natural source, or a combination of the foregoing. The antigen may be purified, partially purified, or a crude extract.

Polypeptide antigens may be isolated from natural sources using standard methods of protein purification known in the art, including, but not limited to, liquid chromatography (e.g., high performance liquid chromatography, fast protein liquid chromatography, etc.), size exclusion chromatography, gel electrophoresis (including one-dimensional gel electrophoresis, two-dimensional gel electrophoresis), affinity chromatography, or other purification technique. In many embodiments, the antigen is a purified antigen, e.g., from about 50% to about 75% pure, from about 75% to about 85% pure, from about 85% to about 90% pure, from about 90% to about 95% pure, from about 95% to about 98% pure, from about 98% to about 99% pure, or greater than 99% pure.

One may employ solid phase peptide synthesis techniques, where such techniques are known to those of skill in the art. See Jones, The Chemical Synthesis of Peptides (Clarendon Press, Oxford) (1994). Generally, in such methods a peptide is produced through the sequential additional of activated monomeric units to a solid phase bound growing peptide chain.

Well-established recombinant DNA techniques can be employed for production of polypeptides either in the same vector as the lipid-raft associated polypeptide, where, e.g., an expression construct comprising a nucleotide sequence encoding a polypeptide is introduced into an appropriate host cell (e.g., a eukaryotic host cell grown as a unicellular entity in in vitro cell culture, e.g., a yeast cell, an insect cell, a mammalian cell, etc.) or a prokaryotic cell (e.g., grown in in vitro cell culture), generating a genetically modified host cell; under appropriate culture conditions, the protein is produced by the genetically modified host cell.

Viral Antigens

Suitable viral antigens include those associated with (e.g., synthesized by) viruses of one or more of the following groups: Retroviridae (e.g. human immunodeficiency viruses, such as HIV-1 (also referred to as HTLV-III, LAV or HTLV-III/LAV, or HIV-III); and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis, including Norwalk and related viruses); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Bimaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); and astroviruses.

Norvirus Antigens

The VLPs disclosed herein may preferably include various antigens from the Norovirus family. Noroviruses, also called “Norwalk-like viruses” represent one of four genera within the Caliciviridae virus family. Within the Norovirus genus there are two major genetic groups that have been designated Genogroup I and Genogroup II. Genogroup I Norovirus strains include Norwalk virus, Southampton virus, Desert Shield virus, and Chiba virus. Genogroup II Norovirus strains include Houston virus, Hawaii virus, Lordsdale virus, Grimsby virus, Mexico virus, and the Snow Mountain agent (Parker, T. D., et al. J Virol. (2005) 79(12):7402-9; Hale, A. D., et al. J Clin. Micro. (2000) 38(4):1656-1660). Norwalk virus (NV) is the prototype strain of a group of human caliciviruses responsible for the majority of epidemic outbreaks of acute viral gastroenteritis worldwide. The Norwalk virus capsid protein has two domains: the shell domain (S) and the protruding domain (P). The P domain (aa 226-530, Norwalk strain numbering) is divided into two subdomains, P1 and P2. The P2 domain is a 127 aa insertion (aa 279-405) in the P1 domain and is located at the most distal surface of the folded monomer. The P2 domain is the least conserved region of VP1 among norovirus strains, and the hypervariable region within P2 is thought to play an important role in receptor binding and immune reactivity. Given the external location of the P domain, it is the preferred antigen or source of polypeptide epitopes for use as antigens for the VLP vaccines disclosed herein. The P2 domain is a preferred antigen for Genogroup I or Genogroup II Norovirus strains. Even more preferred is the mAb 61.21 epitope recently identified as lying in a region of the P2 domain conserved across a range of norovirus strains, as well as the mAb 54.6 epitope (Lochridge, V. P., et al. J Gen. Virol. (2005) 86:2799-2806).

Influenza Antigens

The VLPs disclosed herein may include various antigens from influenza other than, or in addition to, hemagglutinin and neuraminidase. A preferred additional influenza antigen is the M2 polypeptide. The M2 polypeptide of influenza virus is a small 97 amino acid class III integral membrane protein encoded by RNA segment 7 (matrix segment) following a splicing event (80, 81). Very little M2 exists on virus particles but it can be found more abundantly on infected cells. M2 serves as a proton-selective ion channel that is necessary for viral entry (82, 83). It is minimally immunogenic during infection or conventional vaccination, explaining its conservation, but when presented in an alternative format it is more immunogenic and protective (84-86). This is consistent with observations that passive transfer of an M2 monoclonal antibody in vivo accelerates viral clearance and results in protection (87). When the M2 external domain epitope is linked to HBV core particles as a fusion protein it is protective in mice via both parenteral and intranasal inoculation and is most immunogenic when three tandem copies are fused to the N-terminus of the core protein (88-90). This is consistent with other carrier-hapten data showing that increased epitope density increases immunogenicity (91).

For intranasal delivery of an M2 vaccine an adjuvant is required to achieve good protection and good results have been achieved with LTR192G (88, 90) and CTA1-DD (89). The peptide can also be chemically conjugated to a carrier such as KLH, or the outer membrane protein complex of N. meningitides, or human papilloma virus VLPs and is protective as a vaccine in mice and other animals (92, 93).

Insofar as the M2 protein is highly conserved it is not completely without sequence divergence. The M2 ectodomain epitopes of common strains A/PR/8/34 (H1N1) and A/Aichi/68 (H3N2) were shown to be immunologically cross reactive with all other modern sequenced human strains except for A/Hong Kong/156/97 (H5N1)(92). Examination of influenza database sequences also shows similar divergence in the M2 sequence of other more recent pathogenic H5N1 human isolates such as A/Vietnam/1203/04. This finding demonstrates that a successful H5-specific pandemic vaccine incorporating M2 epitopes will need to reflect the M2 sequences that are unique to the pathogenic avian strains rather than M2 sequences currently circulating in human H1 and H3 isolates.

Additional proteins from influenza virus (other than HA, NA and M2) may be included in the VLP vaccine either by co-expression or via linkage of all or part of the additional antigen to the gag or HA polypeptides. These additional antigens include PB2, PB1, PA, nucleoprotein, matrix (M1), NS1, and NS2. These latter antigens are not generally targets of neutralizing antibody responses but may contain important epitopes recognized by T cells. T cell responses induced by a VLP vaccine to such epitopes may prove beneficial in boosting protective immunity.

Other Pathogenic Antigens

Suitable bacterial antigens include antigens associated with (e.g., synthesized by and endogenous to) any of a variety of pathogenic bacteria, including, e.g., pathogenic gram positive bacteria such as pathogenic Pasteurella species, Staphylococci species, and Streptococcus species; and gram-negative pathogens such as those of the genera Neisseria, Escherichia, Bordetella, Campylobacter, Legionella, Pseudomonas, Shigella, Vibrio, Yersinia, Salmonella, Haemophilus, Brucella, Francisella and Bacterioides. See, e.g., Schaechter, M, H. Medoff, D. Schlesinger, Mechanisms of Microbial Disease. Williams and Wilkins, Baltimore (1989)).

Suitable antigens associated with (e.g., synthesized by and endogenous to) infectious pathogenic fungi include antigens associated with infectious fungi including but not limited to: Cryptococcus neoformans, Histoplasma capsulatum, Coccidioides immitis, Blastomyces dermatitidis, and Candida albicans, Candida glabrata, Aspergillus fumigata, Aspergillus flavus, and Sporothrix schenckii.

Suitable antigens associated with (e.g., synthesized by and endogenous to) pathogenic protozoa, helminths, and other eukaryotic microbial pathogens include antigens associated with protozoa, helminths, and other eukaryotic microbial pathogens including, but not limited to, Plasmodium such as Plasmodium falciparum, Plasmodium malariae, Plasmodium ovale, and Plasmodium vivax; Toxoplasma gondii; Trypanosoma brucei, Trypanosoma cruzi; Schistosoma haematobium, Schistosoma mansoni, Schistosoma japonicum; Leishmania donovani; Giardia intestinalis; Cryptosporidium parvum; and the like.

Suitable antigens include antigens associated with (e.g., synthesized by and endogenous to) pathogenic microorganisms such as: Helicobacter pyloris, Borelia burgdorferi, Legionella pneumophila, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Chlamydia trachomatis, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israeli. Non-limiting examples of pathogenic E. coli strains are: ATCC No. 31618, 23505, 43886, 43892, 35401, 43896, 33985, 31619 and 31617.

Any of a variety of polypeptides or other antigens associated with intracellular pathogens may be included in the VLPs. Polypeptides and peptide epitopes associated with intracellular pathogens are any polypeptide associated with (e.g., encoded by) an intracellular pathogen, fragments of which are displayed together with MHC Class I molecule on the surface of the infected cell such that they are recognized by, e.g., bound by a T-cell antigen receptor on the surface of, a CD8.sup.+lymphocyte. Polypeptides and peptide epitopes associated with intracellular pathogens are known in the art and include, but are not limited to, antigens associated with human immunodeficiency virus, e.g., HIV gp120, or an antigenic fragment thereof; cytomegalovirus antigens; Mycobacterium antigens (e.g., Mycobacterium avium, Mycobacterium tuberculosis, and the like); Pneumocystic carinii (PCP) antigens; malarial antigens, including, but not limited to, antigens associated with Plasmodium falciparum or any other malarial species, such as 41-3, AMA-1, CSP, PFEMP-1, GBP-130, MSP-1, PFS-16, SERP, etc.; fungal antigens; yeast antigens (e.g., an antigen of a Candida spp.); toxoplasma antigens, including, but not limited to, antigens associated with Toxoplasma gondii, Toxoplasma encephalitis, or any other Toxoplasma species; Epstein-Barr virus (EBV) antigens; Plasmodium antigens (e.g., gp190/MSP1, and the like); etc.

A preferred VLP vaccine may be directed against Bacillus anthracis. Bacillus anthracis are aerobic or facultative anaerobic Gram-positive, nonmotile rods measuring 1.0 μm wide by 3.0-5.0 μm long. Under adverse conditions, B. anthracis form highly resistant endospores, which can be found in soil at sites where infected animals previously died. A preferred antigen for use in a VLP vaccine as disclosed herein is the protective antigen (PA), an 83 kDa protein that binds to receptors on mammalian cells and is critical to the ability of B. anthracis to cause disease. A more preferred antigen is the C-terminal 140 amino acid fragment of Bacillus anthracis PA which may be used to induce protective immunity in a subject against Bacillus anthracis. Other exemplary antigens for use in a VLP vaccine against anthrax are antigens from the anthrax spore (e.g., BclA), antigens from the vegetative stage of the bacterium (e.g., a cell wall antigen, capsule antigen (e.g., poly-gamma-D-glutamic acid or PGA), secreted antigen (e.g., exotoxin such as protective antigen, lethal factor, or edema factor). Another preferred antigen for use in a VLP vaccine is the tetra-saccharide containing anthrose, which is unique to B. anthracis (Daubenspeck J. M., et al. J. Biol. Chem. (2004), 279:30945). The tetra-saccharide may be coupled to a lipid raft-associated polypeptide allowing association of the antigen with the VLP vaccine.

Tumor-Associated Antigens

Any of a variety of known tumor-specific antigens or tumor-associated antigens (TAA) can be included in the VLPs. The entire TAA may be, but need not be, used. Instead, a portion of a TAA, e.g., an epitope, may be used. Tumor-associated antigens (or epitope-containing fragments thereof) which may be used in VLPs include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, and p21ras. A synthetic analog of any TAA (or epitope thereof), including any of the foregoing, may be used. Furthermore, combinations of one or more TAAs (or epitopes thereof) may be included in the composition.

Allergens

In one aspect, the antigen that is part of the VLP vaccine may be any of a variety of allergens. Allergen based vaccines may be used to induce tolerance in a subject to the allergen. Examples of an allergen vaccine involving co-precipitation with tyrosine may be found in U.S. Pat. Nos. 3,792,159, 4,070,455, and 6,440,426.

Any of a variety of allergens can be included in VLPs. Allergens include but are not limited to environmental aeroallergens; plant pollens such as ragweed/hayfever; weed pollen allergens; grass pollen allergens; Johnson grass; tree pollen allergens; ryegrass; arachnid allergens, such as house dust mite allergens (e.g., Der p I, Der f I, etc.); storage mite allergens; Japanese cedar pollen/hay fever; mold spore allergens; animal allergens (e.g., dog, guinea pig, hamster, gerbil, rat, mouse, etc., allergens); food allergens (e.g., allergens of crustaceans; nuts, such as peanuts; citrus fruits); insect allergens; venoms: (Hymenoptera, yellow jacket, honey bee, wasp, hornet, fire ant); other environmental insect allergens from cockroaches, fleas, mosquitoes, etc.; bacterial allergens such as streptococcal antigens; parasite allergens such as Ascaris antigen; viral antigens; fungal spores; drug allergens; antibiotics; penicillins and related compounds; other antibiotics; whole proteins such as hormones (insulin), enzymes (streptokinase); all drugs and their metabolites capable of acting as incomplete antigens or haptens; industrial chemicals and metabolites capable of acting as haptens and functioning as allergens (e.g., the acid anhydrides (such as trimellitic anhydride) and the isocyanates (such as toluene diisocyanate)); occupational allergens such as flour (e.g., allergens causing Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoas); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherun elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Preferred Methods of Making Enveloped Virus-Based VLPs

Enveloped virus-based VLPs may be made by any method available to one of skill in the art. Enveloped virus-based VLPs typically include one or more polypeptide responsible for the formation of the VLP. In addition, the enveloped virus-based VLP may include one or more additional polypeptide such as a membrane (including lipid-raft)-associated polypeptide to provide (additional) antigens (other than those present naturally or artificially as a part of the one or more polypeptides responsible for the formation of the VLP). In preferred embodiments, the polypeptides may be co-expressed in any available protein expression system, preferably a cell-based system that includes lipid raft domains in the plasma membrane such as mammalian cell expression systems and insect cell expression systems.

Recombinant expression of the polypeptides for the VLPs involves expression vectors containing polynucleotides that encode one or more of the polypeptides. Once a polynucleotide encoding one or more of the polypeptides has been obtained, the vector for the production of the polypeptide may be produced by recombinant DNA technology using techniques well known in the art. Thus, methods for preparing a protein by expressing a polynucleotide containing any of the VLP polypeptide-encoding nucleotide sequences are described herein. Methods which are well known to those skilled in the art can be used to construct expression vectors containing the VLP polypeptide coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a gag polypeptide and a lipid-raft associated polypeptide linked to antigen, all operably linked to one or more promoters.

The expression vector may be transferred to a host cell by conventional techniques and the transfected cells are then cultured by conventional techniques to produce the VLP polypeptide(s). Thus, the invention includes host cells containing a polynucleotide encoding one or more of the VLP polypeptides operably linked to a heterologous promoter. In preferred embodiments for the generation of VLPs, vectors encoding both the gag polypeptide and a lipid-raft associated polypeptide linked to an antigen may be co-expressed in the host cell for generation of the VLP, as detailed below.

A variety of host-expression vector systems may be utilized to express the VLP polypeptides. Such host-expression systems represent vehicles by which the VLP polypeptides may be produced to generate VLPs preferably by co-expression. A wide range of hosts may be used in construct of appropriate expression vectors and, when relying upon lipid-raft based assembly, preferred host-expression systems are those hosts that have lipid rafts suitable for assembly of the VLP. These include but are not limited to microorganisms such as bacteria (e.g., E. coli, B. subtilis) transformed with recombinant bacteriophage DNA, plasmid DNA or cosmid DNA expression vectors containing VLP polypeptide coding sequences; yeast (e.g., Saccharomyces, Pichia) transformed with recombinant yeast expression vectors containing VLP polypeptide coding sequences; insect cell systems infected with recombinant virus expression vectors (e.g., baculovirus) containing VLP polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing VLP polypeptide coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, mammalian cells and more preferably insect cells are used for the expression of the VLP polypeptides, as both have raft lipid suitable for assembly of the VLPs. For example, mammalian cells such as MRC-5 cells, Vero cells, PER.C6™ cells, Chinese hamster ovary cells (CHO), and HEK293 cells, in conjunction with a vector such as the major intermediate early gene promoter element from human cytomegalovirus is an effective expression system for VLP polypeptides (Foecking et al., Gene 45:101 (1986); Cockett et al., Bio/Technology 8:2 (1990)).

In an insect system, Autographa californica nuclear polyhedrosis virus (AcNPV) may be used as a vector to express foreign genes. The virus grows in Spodoptera frugiperda cells. The VLP polypeptide coding sequence(s) may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the VLP polypeptide sequence(s) of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the VLP polypeptide(s) in infected hosts. (e.g., see Logan & Shenk, Proc. Natl. Acad. Sci. USA 81:355-359 (1984)). Specific initiation signals may also be required for efficient translation of inserted VLP polypeptide coding sequence(s). These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)). One example would be the human CMV immediate early promoter as used in adenovirus-based vector systems such as the AdEASY-XL™ system from Stratagene.

In addition, a host cell strain may be chosen which modulates the expression of the inserted sequences, or modifies and processes the gene product in the specific fashion desired. Such modifications (e.g., glycosylation) and processing (e.g., cleavage or transport to the membrane) of protein products may be important for the generation of the VLP or function of a VLP polypeptide or additional polypeptide such as an adjuvant or additional antigen. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

The host cell may be co-transfected with two expression vectors of the invention, the first vector encoding a gag polypeptide and the second vector encoding a viral membrane antigen or a lipid-raft associated polypeptide linked to an antigen. The two vectors may contain identical selectable markers which enable equal expression of each VLP polypeptide. Alternatively, a single vector may be used which encodes, and is capable of expressing, both the gag polypeptide and the lipid-raft associated polypeptide linked to an antigen

Once a VLP has been produced by a host cell, it may be purified by any method known in the art for purification of a polypeptide, for example, by chromatography (e.g., ion exchange, affinity, particularly by affinity for any affinity purification tags added to the polypeptide, and size exclusion chromatography), centrifugation, differential solubility, or by any other standard technique for the purification of proteins or other macromolecules. In addition, the VLP polypeptide can be fused to heterologous polypeptide sequences described herein or otherwise known in the art, to facilitate purification of the VLP. After purification, additional elements such as additional antigens or adjuvants may be physically linked to the VLP either through covalent linkage to the VLP polypeptides or by other non-covalent linkages mechanism. In preferred embodiments where the VLP polypeptides are co-expressed in a host cell that has lipid-raft domains such as mammalian cells and insect cells, the VLPs will self assemble and release allowing purification of the VLPs by any of the above methods. Preferred embodiments of VLPs include VLPs engineered from homologous virus proteins, for example VLPs constructed from M1, HA and optionally NA from influenza virus, and VLPs engineered from heterologous viruses, for example Gag protein from MLV or HIV or other retroviruses engineered to form VLPs with antigens from a different virus, for example influenza HA and NA.

Preferred Methods of Making Gag-Based VLPs

VLPs may be readily assembled by any methods available to one of skill in the art that preferably results in assembled VLPs including a gag polypeptide and a lipid-raft associated polypeptide linked to an antigen which does not naturally associate with a lipid raft. In preferred embodiments, the polypeptides may be co-expressed in any available protein expression system, preferably a cell-based system that includes raft-lipid domains in the lipids such as mammalian cell expression systems and insect cell expression systems.

Numerous examples of expression of VLPs formed using a gag polypeptide have been published demonstrating the range of expression systems available for generating VLPs. Studies with several retroviruses have demonstrated that the Gag polypeptide expressed in the absence of other viral components is sufficient for VLP formation and budding at the cell surface (Wills and Craven AIDS 5, 639-654, 1991; Zhou et al., 3. Virol. 68, 2556-2569, 1994; Morikawa et al., Virology 183, 288-297, 1991; Royer et al., Virology 184, 417-422, 1991; Gheysen et al., Cell 59, 103-112, 1989; Hughes et al., Virology 193, 242-255, 1993; Yamshchikov et al., Virology 214, 50-58, 1995). Formation of VLP upon expression of the Gag precursor in insect cells using a Baculovirus vector has been demonstrated by several groups (Delchambre et al., EMBO J. 8, 2653-2660, 1989; Luo et al., Virology 179, 874-880, 1990; Royer et al., Virology 184, 417-422, 1991; Morikawa et al., Virology 183, 288-297, 1991; Zhou et al., J. Virol. 68, 2556-2569, 1994; Gheysen et al., Cell 59, 103-112, 1989; Hughes et al., Virology 193, 242-255, 1993; Yamshchikov et al., Virology 214, 50-58, 1995). These VLPs resemble immature lentivirus particles and are efficiently assembled and released by budding from the insect cell plasma membrane.

It has been reported that the amino terminal region of the Gag precursor is a targeting signal for transport to the cell surface and membrane binding which is required for virus assembly (Yu et al., J. Virol. 66, 4966-4971, 1992; an, X et al., J. Virol. 67, 6387-6394, 1993; Zhou et al., J. Virol. 68, 2556-2569, 1994; Lee and Linial J. Virol. 68, 6644-6654, 1994; Dorfman et al., J. Virol. 68, 1689-1696, 1994; Facke et al., J. Virol. 67, 4972-4980, 1993). Assembly of recombinant HIV based VLPs that contain Gag structural proteins as well as Env glycoproteins gp120 and gp41 has been reported using a vaccinia virus expression system (Haffar et al., J. Virol. 66, 4279-4287, 1992).

Preferred Methods of Inactivation of Infectious Agents in Enveloped Virus-Based VLP Preparations

The preferred method of inactivation is through electromagnetic radiation as electromagnetic radiation is capable of inactivating the infectious agents without substantially reducing the immunogenicity of the enveloped virus-based VLP. As all three preferred modes of electromagnetic radiation (i.e, UV irradiation with photoreactive compounds, UV irradiation alone and gamma irradiation) have a long history of use for inactivation of pathogens in a wide variety of samples such as blood, food, vaccines, etc. there are a wide variety of commercially available apparatus for applying the inactivating electromagnetic radiation that may be used with little to no modification to practice the methods disclosed herein. Furthermore, optimizing wavelengths and dosages is routine in the art and therefore readily within the capabilities of one of ordinary skill in the art.

UV Irradiation with Photoreactive Compounds

An exemplary method of inactivation with electromagnetic radiation is a combination of ultraviolet irradiation, such as UV-A irradiation, in the presence of a photoreactive compound, preferable one that will react with polynucleotides in the infectious agent.

Preferred photoreactive compounds include: actinomycins, anthracyclinones, anthramycin, benzodipyrones, fluorenes, fluorenones, furocoumarins, isoalloxazine, mitomycin, monostral fast blue, norphillin A, phenanthridines, phenazathionium salts, phenazines, phenothiazines, phenylazides, quinolines, and thiaxanthenones. A preferred species are furocoumarins which belong in one of two main categories. The first category is psoralens [7H-furo(3,2-g)-(1)-benzopyran-7-one, or delta-lactone of 6-hydroxy-5-benzofuranacrylic acid], which are linear and in which the two oxygen residues appended to the central aromatic moiety have a 1, 3 orientation, and further in which the furan ring moiety is linked to the 6 position of the two ring coumarin system. The second category is isopsoralens [2H-furo(2,3-h)-(1)-benzopyran-2-one, or delta-lactone of 4-hydroxy-5-benzofuranacrylic acid], which are angular and in which the two oxygen residues appended to the central aromatic moiety have a 1, 3 orientation, and further in which the furan ring moiety is linked to the 8 position of the two ring coumarin system. Psoralen derivatives may be generated by substitution of the linear furocoumarin at the 3, 4, 5, 8, 4′, or 5′ positions, while isopsoralen derivatives may be generated by substitution of the angular furocoumarin at the 3, 4, 5, 6, 4′, or 5 positions. Psoralens can intercalate between the base pairs of double-stranded nucleic acids, forming covalent adducts to pyrimidine bases upon absorption of long wave ultraviolet light (UVA). See, e.g., G. D. Cimino et al., Ann. Rev. Biochem. 54:1151 (1985); Hearst et al., Quart. Rev. Biophys. 17:1 (1984).

The wavelengths of the preferred UV (or in some cases visible light) radiation will depend upon the wavelength at which appropriate reactions and/or photoadducts are generated which is dependent upon the chemistry of the photoreactive chemical. By way of example, UV radiation in the wavelengths between 320 and 380 nm are most effective for many psoralens with 330 to 360 nm having maximum effectiveness. Similar UV-A wavelengths are also highly effective in conjunction with riboflavin, a photoreactive compound that can also be used coupled with visible light such as 419 nm for pathogen inactivation.

UV Irradiation Alone

In addition to UV irradiation in the presence of a photoreactive compound, infectious agents may be inactivated by UV irradiation alone. In a preferred embodiment, the radiation is UV-C radiation having a wavelength between about 180 and 320 nm, or between about 225 and 290 nm, or about 254 nm (i.e., spectral region with a high absorbance peak of polynucleotides and diminished protein absorption). UV-C radiation is preferred because it is less detrimental to the components of the enveloped virus-based VLPs disclosed herein for both stability and immunogenicity such as the lipid bilayer forming the envelope and proteins within the envelope while retaining sufficient energy to inactivate infectious agents. However, other types of UV radiation such as, for example, UV-A and UV-B may also be used.

Gamma Irradiation

Gamma irradiation (i.e., ionizing radiation) may also be used in the practice of the methods disclosed herein to generate the compositions. In this preferred embodiment, gamma irradiation doses of between 10 and 60 kGy are effective for pathogen inactivation. Gamma irradiation can directly inactivate infectious agents by introducing strand breaks in the polynucleotides encoding the genome of the infectious agent or indirectly by generating free radicals that attack the polynucleotides. Free radical scavengers and low temperature may be used in conjunction with gamma irradiation to inhibit radical-mediated damage to lipid and protein components of enveloped VLPs.

Preferred Methods of Using Enveloped Virus-Based VLPs

Formulations

A preferred use of the enveloped virus-based VLPs described herein is as a vaccine preparation. Typically, such vaccines are prepared as injectables either as liquid solutions or suspensions; solid forms suitable for solution in, or suspension in, liquid prior to injection may also be prepared. Such preparations may also be emulsified or produced as a dry powder. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain auxiliary substances such as wetting or emulsifying agents, pH buffering agents, or adjuvants which enhance the effectiveness of the vaccines.

Vaccines may be conventionally administered parenterally, by injection, for example, either subcutaneously, intradermally, subdermally or intramuscularly. Additional formulations which are suitable for other modes of administration include suppositories and, in some cases, oral, intranasal, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. In certain embodiments, a low melting wax, such as a mixture of fatty acid glycerides or cocoa butter is first melted and the enveloped virus-based VLPs described herein are dispersed homogeneously, for example, by stirring. The molten homogeneous mixture is then poured into conveniently sized molds, allowed to cool, and to solidify.

Formulations suitable for intranasal delivery include liquids and dry powders. Formulations include such normally employed excipients as, for example, pharmaceutical grades of mannitol, lactose, sucrose, trehalose, and chitosan. Mucosadhesive agents such as chitosan can be used in either liquid or powder formulations to delay mucocilliary clearance of intranasally-administered formulations. Sugars such as mannitol and sucrose can be used as stability agents in liquid formulations and as stability and bulking agents in dry powder formulations. In addition, adjuvants such as monophosphoryl lipid A (MPL) can be used in both liquid and dry powder formulations as an immunostimulatory adjuvant.

Formulations suitable for oral delivery include liquids, solids, semi-solids, gels, tablets, capsules, lozenges, and the like. Formulations suitable for oral delivery include tablets, lozenges, capsules, gels, liquids, food products, beverages, nutraceuticals, and the like. Formulations include such normally employed excipients as, for example, pharmaceutical grades of mannitol, lactose, starch, magnesium stearate, sodium saccharine, cellulose, magnesium carbonate, and the like. Other enveloped virus-based VLP vaccine compositions may take the form of solutions, suspensions, pills, sustained release formulations or powders and contain 10-95% of active ingredient, preferably 25-70%. For oral formulations, cholera toxin is an interesting formulation partner (and also a possible conjugation partner).

The enveloped virus-based VLP vaccines when formulated for vaginal administration may be in the form of pessaries, tampons, creams, gels, pastes, foams or sprays. Any of the foregoing formulations may contain agents in addition to enveloped virus-based VLPs, such as carriers, known in the art to be appropriate.

In some embodiments, the enveloped virus-based VLP vaccine may be formulated for systemic or localized delivery. Such formulations are well known in the art. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Systemic and localized routes of administration include, e.g., intradermal, topical application, intravenous, intramuscular, etc.

The enveloped virus-based VLPs may be formulated into the vaccine including neutral or salt-based formulations. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines may be administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2000 μg (even though higher amounts in the 1-10 mg range are contemplated), such as in the range from about 0.5 μg to 1000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the vaccine formulations will be sufficiently immunogenic as a vaccine by themselves, but for some of the others the immune response will be enhanced if the vaccine further includes an adjuvant substance.

Delivery agents that improve mucoadhesion can also be used to improve delivery and immunogenicity especially for intranasal, oral or lung based delivery formulations. One such compound, chitosan, the N-deacetylated form of chitin, is used in many pharmaceutical formulations (32). It is an attractive mucoadhesive agent for intranasal vaccine delivery due to its ability to delay mucociliary clearance and allow more time for mucosal antigen uptake and processing (33, 34). In addition, it can transiently open tight junctions which may enhance transepithelial transport of antigen to the NALT. In a recent human trial, a trivalent inactivated influenza vaccine administered intranasally with chitosan but without any additional adjuvant yielded seroconversion and HI titers that were only marginally lower than those obtained following intramuscular inoculation (33).

Chitosan can also be formulated with adjuvants that function well intranasally such as the genetically detoxified E. coli heat-labile enterotoxin mutant LTK63. This adds an immunostimulatory effect on top of the delivery and adhesion benefits imparted by chitosan resulting in enhanced mucosal and systemic responses (35).

Finally, it should be noted that chitosan formulations can also be prepared in a dry powder format that has been shown to improve vaccine stability and result in a further delay in mucociliary clearance over liquid formulations (42). This was seen in a recent human clinical trial involving an intranasal dry powder diphtheria toxoid vaccine formulated with chitosan in which the intranasal route was as effective as the traditional intramuscular route with the added benefit of secretory IgA responses (43). The vaccine was also very well tolerated. Intranasal dry powdered vaccines for anthrax containing chitosan and MPL induce stronger responses in rabbits than intramuscular inoculation and are also protective against aerosol spore challenge (44).

Intranasal vaccines represent a preferred formulation as they can affect the upper and lower respiratory tracts in contrast to parenterally administered vaccines which are better at affecting the lower respiratory tract. This can be beneficial for inducing tolerance to allergen-based vaccines and inducing immunity for pathogen-based vaccines.

In addition to providing protection in both the upper and lower respiratory tracts, intranasal vaccines avoid the complications of needle inoculations and provide a means of inducing both mucosal and systemic humoral and cellular responses via interaction of particulate and/or soluble antigens with nasopharyngeal-associated lymphoid tissues (NALT) (16-19). The intranasal route has been historically less effective than parenteral inoculation, but the use of enveloped virus-based VLPs, novel delivery formulations, and adjuvants are beginning to change the paradigm. Indeed, enveloped virus-based VLPs containing functional hemagglutinin polypeptides may be especially well suited for intranasal delivery due to the abundance of sialic acid-containing receptors in the nasal mucosa resulting in the potential for enhanced HA antigen binding and reduced mucociliary clearance.

Adjuvants

Various methods of achieving adjuvant effect for vaccines are known and may be used in conjunction with the enveloped virus-based VLPs disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9.

In some embodiments, a enveloped virus-based VLP vaccine includes the enveloped virus-based VLP in admixture with at least one adjuvant, at a weight-based ratio of from about 10:1 to about 10¹⁰:1 enveloped virus-based VLP:adjuvant, e.g., from about 10:1 to about 100:1, from about 100:1 to about 10³:1, from about 10³:1 to about 10⁴:1, from about 10⁴:1 to about 10⁵:1, from about 10⁵:1 to about 10⁶:1, from about 10⁶:1 to about 10⁷:1, from about 10⁷:1 to about 10⁸:1, from about 10⁸:1 to about 10⁹:1, or from about 10⁹:1 to about 10¹⁰:1 enveloped virus-based VLP:adjuvant. One of skill in the art can readily determine the appropriate ratio through information regarding the adjuvant and routine experimentation to determine optimal ratios.

Preferred examples of adjuvants are polypeptide adjuvants that may be readily added to the enveloped virus-based VLPs described herein by co-expression with the polypeptide component of the enveloped virus-based VLP or fusion with the polypeptide component to produce chimeric polypeptides. Bacterial flagellin, the major protein constituent of flagella, is a preferred adjuvant which has received increasing attention as an adjuvant protein because of its recognition by the innate immune system by the toll-like receptor TLR5 (65). Flagellin signaling through TLR5 has effects on both innate and adaptive immune functions by inducing DC maturation and migration as well as activation of macrophages, neutrophils, and intestinal epithelial cells resulting in production of proinflammatory mediators (66-72).

TLR5 recognizes a conserved structure within flagellin monomers that is unique to this protein and is required for flagellar function, precluding its mutation in response to immunological pressure (73). The receptor is sensitive to a 100 fM concentration but does not recognize intact filaments. Flagellar disassembly into monomers is required for binding and stimulation.

As an adjuvant, flagellin has potent activity for induction of protective responses for heterologous antigens administered either parenterally or intranasally (66, 74-77) and adjuvant effects for DNA vaccines have also been reported (78). A Th2 bias is observed when flagellin is employed which would be appropriate for a respiratory virus such as influenza but no evidence for IgE induction in mice or monkeys has been observed. In addition, no local or systemic inflammatory responses have been reported following intranasal or systemic administration in monkeys (74). The Th2 character of responses elicited following use of flagellin is somewhat surprising since flagellin signals through TLR5 in a MyD88-dependent manner and all other MyD88-dependent signals through TLRs have been shown to result in a Th1 bias (67, 79). Importantly, pre-existing antibodies to flagellin have no appreciable effect on adjuvant efficacy (74) making it attractive as a multi-use adjuvant.

A common theme in many recent intranasal vaccine trials is the use of adjuvants and/or delivery systems to improve vaccine efficacy. In one such study an influenza H3 vaccine containing a genetically detoxified E. coli heat-labile enterotoxin adjuvant (LT R192G) resulted in heterosubtypic protection against H5 challenge but only following intranasal delivery. Protection was based on the induction of cross neutralizing antibodies and demonstrated important implications for the intranasal route in development of new vaccines (22).

Cytokines, colony-stimulating factors (e.g., GM-CSF, CSF, and the like); tumor necrosis factor; interleukin-2, -7, -12, interferons and other like growth factors, may also be used as adjuvants and are also preferred as they may be readily included in the enveloped virus-based VLP vaccine by admixing or fusion with the polypeptide component.

In some embodiments, the enveloped virus-based VLP vaccine compositions disclosed herein may include other adjuvants that act through a Toll-like receptor such as a nucleic acid TLR9 ligand comprising a 5′-TCG-3′ sequence; an imidazoquinoline TLR7 ligand; a substituted guanine TLR7/8 ligand; other TLR7 ligands such as Loxoribine, 7-deazadeoxyguanosine, 7-thia-8-oxodeoxyguanosine, Imiquimod (R-837), and Resiquimod (R-848).

Certain adjuvants facilitate uptake of the vaccine molecules by APCs, such as dendritic cells, and activate these. Non-limiting examples are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; MPL; and an encapsulating adjuvant.

Additional examples of adjuvants include agents such as aluminum salts such as hydroxide or phosphate (alum), commonly used as 0.05 to 0.1 percent solution in buffered saline (see, e.g., Nicklas (1992) Res. Immunol. 143:489-493), admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

DDA (dimethyldioctadecylammonium bromide) is an interesting candidate for an adjuvant, but also Freund's complete and incomplete adjuvants as well as quillaja saponins such as QuilA and QS21 are interesting. Further possibilities include poly[di(earboxylatophenoxy)phosphazene (PCPP) derivatives of lipopolysaccharides such as monophosphoryl lipid A (MPL®), muramyl dipeptide (MDP) and threonyl muramyl dipeptide (tMDP). The lipopolysaccharide based adjuvants are preferred for producing a predominantly Th1-type response including, for example, a combination of monophosphoryl lipid A, preferably 3-de-O-acylated monophosphoryl lipid A, together with an aluminum salt. MPL® adjuvants are available from GlaxoSmithKline (see, for example, U.S. Pat. Nos. 4,436,727; 4,877,611; 4,866,034 and 4,912,094).

Liposome formulations are also known to confer adjuvant effects, and therefore liposome adjuvants are preferred examples in conjunction with the enveloped virus-based VLPs.

Immunostimulating complex matrix type (ISCOM® matrix) adjuvants are preferred choices according to the invention, especially since it has been shown that this type of adjuvants are capable of up-regulating MHC Class II expression by APCs. An ISCOM matrix consists of (optionally fractionated) saponins (triterpenoids) from Quillaja saponaria, cholesterol, and phospholipid. When admixed with the immunogenic protein such as in the VLPs, the resulting particulate formulation is what is known as an ISCOM particle where the saponin may constitute 60-70% w/w, the cholesterol and phospholipid 10-15% w/w, and the protein 10-15% w/w. Details relating to composition and use of immunostimulating complexes can for example be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 provide useful instructions for the preparation of complete immunostimulating complexes.

The saponins, whether or not in the form of iscoms, that may be used in the adjuvant combinations with the enveloped virus-based VLP vaccines disclosed herein include those derived from the bark of Quillaja Saponaria Molina, termed Quil A, and fractions thereof, described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particularly preferred fractions of Quil A are QS21, QS7, and QS17.

β-Escin is another preferred haemolytic saponins for use in the adjuvant compositions of the present invention. Escin is described in the Merck index (12th ed: entry 3737) as a mixture of saponins occurring in the seed of the horse chestnut tree, Lat: Aesculus hippocastanum. Its isolation is described by chromatography and purification (Fiedler, Arzneimittel-Forsch. 4, 213 (1953)), and by ion-exchange resins (Erbring et al., U.S. Pat. No. 3,238,190). Fractions of escin have been purified and shown to be biologically active (Yoshikawa M, et al. (Chem Pharm Bull (Tokyo) 1996 August; 44(8):1454-1464)). β-escin is also known as aescin.

Another preferred haemolytic saponin for use in the present invention is Digitonin. Digitonin is described in the Merck index (12.sup.th Edition, entry 3204) as a saponin, being derived from the seeds of Digitalis purpurea and purified according to the procedure described Gisvold et al., J. Am. Pharm. Assoc., 1934, 23, 664; and Ruhenstroth-Bauer, Physiol. Chem., 1955, 301, 621. Its use is described as being a clinical reagent for cholesterol determination.

Another interesting (and thus, preferred) possibility of achieving adjuvant effect is to employ the technique described in Gosselin et al., 1992. In brief, the presentation of a relevant antigen such as an antigen of the present invention can be enhanced by conjugating the antigen to antibodies (or antigen binding antibody fragments) against the F_{C} receptors on monocytes/macrophages. Especially conjugates between antigen and anti-F_{C}RI have been demonstrated to enhance immunogenicity for the purposes of vaccination. The antibody may be conjugated to the enveloped virus-based VLP after generation or as a part of the generation including by expressing as a fusion to any one of the polypeptide components of the enveloped virus-based VLP.

Other possibilities involve the use of the targeting and immune modulating substances (i.e. cytokines). In addition, synthetic inducers of cytokines such as poly I:C may also be used.

Suitable mycobacterial derivatives may be selected from the group consisting of muramyl dipeptide, complete Freund's adjuvant, RIBI, (Ribi ImmunoChem Research Inc., Hamilton, Mont.) and a diester of trehalose such as TDM and TDE.

Examples of suitable immune targeting adjuvants include CD40 ligand and CD40 antibodies or specifically binding fragments thereof (cf. the discussion above), mannose, a Fab fragment, and CTLA-4.

Examples of suitable polymer adjuvants include a carbohydrate such as dextran, PEG, starch, mannan, and mannose; a plastic polymer; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12-15, 1998, Seascape Resort, Aptos, Calif.”

Oligonucleotides may be used as adjuvants in conjunction with the enveloped virus-based VLP vaccines and preferably contain two or more dinucleotide CpG motifs separated by at least three or more preferably at least six or more nucleotides. CpG-containing oligonucleotides (in which the CpG dinucleotide is unmethylated) induce a predominantly Th1 response. Such oligonucleotides are well known and are described, for example, in WO 96/02555, WO 99/33488 and U.S. Pat. Nos. 6,008,200 and 5,856,462.

Such oligonucleotide adjuvants may be deoxynucleotides. In a preferred embodiment the nucleotide backbone in the oligonucleotide is phosphorodithioate, or more preferably a phosphorothioate bond, although phosphodiester and other nucleotide backbones such as PNA are within the scope of the invention including oligonucleotides with mixed backbone linkages. Methods for producing phosphorothioate oligonucleotides or phosphorodithioate are described in U.S. Pat. No. 5,666,153, U.S. Pat. No. 5,278,302 and W095/26204.

Examples of preferred oligonucleotides have the following sequences. The sequences preferably contain phosphorothioate modified nucleotide backbones.

(SEQ ID NO: 1)
OLIGO 1: TCC ATG ACG TTC CTG ACG TT (CpG 1826)
(SEQ ID NO: 2)
OLIGO 2: TCT CCC AGC GTG CGC CAT (CpG 1758)
(SEQ ID NO: 3)
OLIGO 3: ACC GAT GAC GTC GCC GGT GAC GGC ACC ACG
(SEQ ID NO: 4)
OLIGO 4: TCG TCG TTT TGT CGT TTT GTC GTT (CpG 2006)
(SEQ ID NO: 5)
OLIGO 5: TCC ATG ACG TTC CTG ATG CT (CpG 1668)

Alternative preferred CpG oligonucleotides include the above sequences with inconsequential deletions or additions thereto. The CpG oligonucleotides as adjuvants may be synthesized by any method known in the art (e.g., EP 468520). Preferably, such oligonucleotides may be synthesized utilizing an automated synthesizer. Such oligonucleotide adjuvants may be between 10-50 bases in length. Another adjuvant system involves the combination of a CpG-containing oligonucleotide and a saponin derivative particularly the combination of CpG and QS21 is disclosed in WO 00/09159.

Many single or multiphase emulsion systems have been described. One of skill in the art may readily adapt such emulsion systems for use with enveloped virus-based VLPs so that the emulsion does not disrupt the enveloped virus-based VLP's structure. Oil in water emulsion adjuvants per se have been suggested to be useful as adjuvant compositions (EPO 399 843B), also combinations of oil in water emulsions and other active agents have been described as adjuvants for vaccines (WO 95/17210; WO 98/56414; WO 99/12565; WO 99/11241). Other oil emulsion adjuvants have been described, such as water in oil emulsions (U.S. Pat. No. 5,422,109; EP 0 480 982 B2) and water in oil in water emulsions (U.S. Pat. No. 5,424,067; EP 0 480 981 B).

The oil emulsion adjuvants for use with the enveloped virus-based VLP vaccines described herein may be natural or synthetic, and may be mineral or organic. Examples of mineral and organic oils will be readily apparent to the man skilled in the art.

In order for any oil in water composition to be suitable for human administration, the oil phase of the emulsion system preferably includes a metabolizable oil. The meaning of the term metabolizable oil is well known in the art. Metabolizable can be defined as “being capable of being transformed by metabolism” (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts (such as peanut oil), seeds, and grains are common sources of vegetable oils. Synthetic oils are also part of this invention and can include commercially available oils such as NEOBEE® and others. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolizable oil virtue of the fact that it is an intermediate in the biosynthesis of cholesterol (Merck index, 10th Edition, entry no. 8619).

Particularly preferred oil emulsions are oil in water emulsions, and in particular squalene in water emulsions.

In addition, the most preferred oil emulsion adjuvants of the present invention include an antioxidant, which is preferably the oil α-tocopherol (vitamin E, EP 0 382 271 B1).

WO 95/17210 and WO 99/11241 disclose emulsion adjuvants based on squalene, α-tocopherol, and TWEEN 80, optionally formulated with the immunostimulants QS21 and/or 3D-MPL. WO 99/12565 discloses an improvement to these squalene emulsions with the addition of a sterol into the oil phase. Additionally, a triglyceride, such as tricaprylin (C27H50O6), may be added to the oil phase in order to stabilize the emulsion (WO 98/56414).

The size of the oil droplets found within the stable oil in water emulsion are preferably less than 1 micron, may be in the range of substantially 30-600 nm, preferably substantially around 30-500 nm in diameter, and most preferably substantially 150-500 nm in diameter, and in particular about 150 nm in diameter as measured by photon correlation spectroscopy. In this regard, 80% of the oil droplets by number should be within the preferred ranges, more preferably more than 90% and most preferably more than 95% of the oil droplets by number are within the defined size ranges. The amounts of the components present in the oil emulsions of the present invention are conventionally in the range of from 2 to 10% oil, such as squalene; and when present, from 2 to 10% alpha tocopherol; and from 0.3 to 3% surfactant, such as polyoxyethylene sorbitan monooleate. Preferably the ratio of oil:alpha tocopherol is equal or less than 1 as this provides a more stable emulsion. Span 85 may also be present at a level of about 1%. In some cases it may be advantageous that the enveloped virus-based VLP vaccines disclosed herein will further contain a stabilizer.

The method of producing oil in water emulsions is well known to the man skilled in the art. Commonly, the method includes the step of mixing the oil phase with a surfactant such as a PBS/TWEEN80® solution, followed by homogenization using a homogenizer, it would be clear to a man skilled in the art that a method comprising passing the mixture twice through a syringe needle would be suitable for homogenizing small volumes of liquid. Equally, the emulsification process in microfluidizer (M110S microfluidics machine, maximum of 50 passes, for a period of 2 minutes at maximum pressure input of 6 bar (output pressure of about 850 bar)) could be adapted by the man skilled in the art to produce smaller or larger volumes of emulsion. This adaptation could be achieved by routine experimentation comprising the measurement of the resultant emulsion until a preparation was achieved with oil droplets of the required diameter.

The enveloped virus-based VLP vaccine preparations disclosed herein may be used to protect or treat a mammal or bird susceptible to, or suffering from a viral infection, by means of administering the vaccine by intranasal, intramuscular, intraperitoneal, intradermal, transdermal, intravenous, or subcutaneous administration. Methods of systemic administration of the vaccine preparations may include conventional syringes and needles, or devices designed for ballistic delivery of solid vaccines (WO 99/27961), or needleless pressure liquid jet device (U.S. Pat. No. 4,596,556; U.S. Pat. No. 5,993,412), or transdermal patches (WO 97/48440; WO 98/28037). The enveloped virus-based VLP vaccines may also be applied to the skin (transdermal or transcutaneous delivery WO 98/20734; WO 98/28037). The enveloped virus-based VLP vaccines disclosed herein therefore includes a delivery device for systemic administration, pre-filled with the enveloped virus-based VLP vaccine or adjuvant compositions. Accordingly there is provided a method for inducing an immune response in an individual preferably mammal or bird, comprising the administration of a vaccine comprising any of the enveloped virus-based VLP compositions described herein and optionally including an adjuvant and/or a carrier, to the individual, wherein the vaccine is administered via the parenteral or systemic route.

Preferably the vaccine preparations of the present invention may be used to protect or treat a mammal or bird susceptible to, or suffering from a viral infection, by means of administering the vaccine via a mucosal route, such as the oral/alimentary or nasal route. Alternative mucosal routes are intravaginal and intra-rectal. The preferred mucosal route of administration is via the nasal route, termed intranasal vaccination. Methods of intranasal vaccination are well known in the art, including the administration of a droplet, spray, or dry powdered form of the vaccine into the nasopharynx of the individual to be immunized. Nebulized or aerosolized vaccine formulations are therefore preferred forms of the enveloped virus-based VLP vaccines disclosed herein. Enteric formulations such as gastro resistant capsules and granules for oral administration, suppositories for rectal or vaginal administration are also formulations of the enveloped virus-based VLP vaccines disclosed herein.

The preferred enveloped virus-based VLP vaccine compositions disclosed herein, represent a class of mucosal vaccines suitable for application in humans to replace systemic vaccination by mucosal vaccination.

The enveloped virus-based VLP vaccines may also be administered via the oral route. In such cases the pharmaceutically acceptable excipient may also include alkaline buffers, or enteric capsules or microgranules. The enveloped virus-based VLP vaccines may also be administered by the vaginal route. In such cases, the pharmaceutically acceptable excipients may also include emulsifiers, polymers such as CARBOPOL®, and other known stabilizers of vaginal creams and suppositories. The enveloped virus-based VLP vaccines may also be administered by the rectal route. In such cases the excipients may also include waxes and polymers known in the art for forming rectal suppositories.

Alternatively the enveloped virus-based VLP vaccines formulations may be combined with vaccine vehicles composed of chitosan (as described above) or other polycationic polymers, polylactide and polylactide-coglycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM.

Additional illustrative adjuvants for use in the pharmaceutical and vaccine compositions using enveloped virus-based VLPs as described herein include SAF (Chiron, Calif., United States), MF-59 (Chiron, see, e.g., Granoff et al. (1997) Infect Immun. 65 (5):1710-1715), the SBAS series of adjuvants (e.g., SB-AS2 (SmithKline Beecham adjuvant system #2; an oil-in-water emulsion containing MPL and QS21); SBAS-4 (SmithKline Beecham adjuvant system #4; contains alum and MPL), available from SmithKline Beecham, Rixensart, Belgium), Detox (Enhanzyn®) (GlaxoSmithKline), RC-512, RC-522, RC-527, RC-529, RC-544, and RC-560 (GlaxoSmithKline) and other aminoalkyl glucosaminide 4-phosphates (AGPs), such as those described in pending U.S. patent application Ser. Nos. 08/853,826 and 09/074,720.

Other examples of adjuvants include, but are not limited to, Hunter's TiterMax® adjuvants (CytRx Corp., Norcross, Ga.); Gerbu adjuvants (Gerbu Biotechnik GmbH, Gaiberg, Germany); nitrocellulose (Nilsson and Larsson (1992) Res. Immunol. 143:553-557); alum (e.g., aluminum hydroxide, aluminum phosphate) emulsion based formulations including mineral oil, non-mineral oil, water-in-oil or oil-in-water emulsions, such as the Seppic ISA series of Montamide adjuvants (e.g., ISA-51, ISA-57, ISA-720, ISA-151, etc.; Seppic, Paris, France); and PROVAX® (IDEC Pharmaceuticals); OM-174 (a glucosamine disaccharide related to lipid A); Leishmania elongation factor; non-ionic block copolymers that form micelles such as CRL 1005; and Syntex Adjuvant Formulation. See, e.g., O'Hagan et al. (2001) Biomol Eng. 18(3):69-85; and “Vaccine Adjuvants: Preparation Methods and Research Protocols” D. O'Hagan, ed. (2000) Humana Press.

Other preferred adjuvants include adjuvant molecules of the general formula

HO(CH₂CH₂O)ₙ-A-R, (I)

wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C.sub.1-50, preferably C.sub.4-C.sub.20 alkyl and most preferably C.sub.12 alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12.sup.th edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described above.

Further examples of suitable pharmaceutically acceptable excipients for use with the enveloped virus-based VLP vaccines disclosed herein include water, phosphate buffered saline, isotonic buffer solutions.

This invention will be better understood by reference to the following non-limiting Examples. As described herein, the invention includes chimeric enveloped virus-based VLPs incorporating any type of lipid raft-associated polypeptide linked to an antigen which does not naturally associate with a lipid raft. The following Examples describe a representative embodiment of the invention, chimeric enveloped virus-based VLPs with influenza antigens.

### Example 1

### Production of Influenza-Pseudotyped Gag VLPs

The MLV Gag gene and the HA and NA genes of various influenza A subtypes were individually cloned into the pFastBac1 baculovirus transfer vector behind the polyhedrin promoter as described below. To construct a “triple gene” expression vector, the complete transcription unit of one HA and one NA vector were excised by cleavage with SnaB I and Hpa I and these blunt end fragments were transferred into unique SnaB I and Hpa I cloning sites, respectively, on either side of the Gag transcription unit in the Gag gene transfer vector. This resulted in a single plasmid containing three separate transcription units (HA, Gag, NA) arranged in a head-to-tail fashion. The pFB-HA-pGag-pNA triple transfer vectors representing various influenza A subtypes were then transformed into DH10Bac cells for recombination into the baculovirus genome as described by the kit manufacturer (Invitrogen, Carlsbad, Calif.).

MLV Gag and Influenza HA and NA Genes:

The Gag gene of murine leukemia virus was derived from the plasmid pAMS (ATCC, Manassas, Va.) by PCR using the following primers: 5′ CACCATGGGCCAGACTGTTACC 3′ (SEQ ID:6) and 5′ CTACTAGTCATCTAGGGTCAGGAG 3′ (SEQ ID:7). The CACC extension at the 5′ end of the first primer facilitated unidirectional insertion into the vector pENTR D-TOPO via Gateway cloning technology (Invitrogen) resulting in the plasmid pENTR-Gag. The Gag coding sequence was confirmed by DNA sequencing then transferred into pFastbac1 as follows: pENTR-Gag was cut with Asc I and the ends were rendered blunt by treatment with Klenow DNA polymerase after which the DNA was cleaved with Not I. The resultant Gag fragment was ligated into pFastbac1 vector DNA after first cleaving pFastbac1 with Sph I, rendering the ends blunt, then cleaving with Not I.

The HA and NA genes of influenza A/PR/8/34 (H1N1) and A/Hong Kong/68 (H3N2) were cloned by RT-PCR. Viral RNA was prepared from egg-grown virus using the QIAmp MinElute Virus Spin Kit (Qiagen, Valencia, Calif.) and first strand cDNA reactions were performed using the Accuscript High Fidelity 1st Strand cDNA Synthesis kit (Stratagene, La Jolla, Calif.). Following first strand cDNA synthesis, standard PCR reactions were employed to amplify the HA and NA fragments. Primers for the PR/8 H1 gene were as follows: 5′ CACCATGAAGGCAAACCTACTGGTCC 3′ (SEQ ID:8) and 5′ TCAGATGCATATTCTGCACTGC 3′ (SEQ ID:9). Primers for the PR/8 N1 gene were as follows: 5′ CACCATGAATCCAAATCAGAAAATAATAACCATTCC 3′ (SEQ ID:10) and 5′ CTACTTGTCAATGGTGAATGGCAAC 3′ (SEQ ID:11). Primers for the A/Hong Kong/68 H3 gene were as follows: 5′ CACCATGAAGACCATCATTGCTTTGAGC 3′ (SEQ ID:12) and 5′ TCAAATGCAAATGTTGCACCTAATGTTGCC 3′ (SEQ ID:13). Primers for the A/Hong Kong/68 N2 gene were as follows: 5′ ATATAGGCGCGCCACCATGAATCCAAATCAAAAGATAATAACAATTGGC 3′ (SEQ ID:14) and 5′ ATATAGCGGCCGCTTATATAGGCATGAAATTGATGTTCGC 3′ (SEQ ID:15). The HA and NA genes of A/PR/8/34 (H1N1) and the HA gene of A/Hong Kong/68 (H3N2) were first cloned into pENTR D-TOPO, confirmed by sequencing, then transferred into pFastbac1 as described above. The NA gene of A/Hong Kong/68 (H3N2) was directly cloned into BssHII-NotI-cut pFastbac1 after trimming the PCR fragment ends with AscI and Not I. Candidate clones were confirmed by sequencing.

Plasmid clones containing the H5 and N1 genes of A/Vietnam/1203/04 and A/Indonesia/5/05 were obtained from Dr. Ruben Donis (Branch Chief, Molecular Virology and Vaccines, CDC, Atlanta, Ga. USA). The H5 clones contained deletions of the poly-basic regions at the maturational cleavage site. The provided plasmids were used as templates to generate “Vietnam” and “Indonesia” H5 and N1 PCR fragments for insertion into pENTR D-TOPO for sequence confirmation. The H5 primers were as follows: 5′ CACCATGGAGAAAATAGTGCTTC 3′ (SEQ ID:16) and 5′ TTAAATGCAAATTCTGCATTGTAACG 3′ (SEQ ID:17). The N1 primers were as follows: 5′ CACCATGAATCCAAATCAGAAGATAATAACC 3′ (SEQ ID:18) and 5′ CTACTTGTCAATGGTGAATGGC 3′ (SEQ ID:19). After sequence confirmation, the individual H5 and N1 genes were transferred into pFastbac1 as describe above.

The HA and NA genes of A/Wisconsin/67/2005 (H3N2) were cloned by RT-PCR from virus RNA as described above. Primers for the H3 gene were as follows: 5′ ATATAGGCGCGCCACCATGAAGACTATCATTGCTTTGAGC 3′ (SEQ ID:20) and 5′ ATATAGCGGCCGCTCAAATGCAAATGTTGCACCTAATGTTGCC 3′ (SEQ ID:21). Primers for the N2 gene were as follows: 5′ ATATAGGCGCGCCACCATGAATCCAAATCAAAAGATAATAACGATTGGC 3′ (SEQ ID:22) and 5′ ATATAGCGGCCGCTTATATAGGCATGAGATTGATGTCCG 3′ (SEQ ID:23). The H3 and N2 gene fragments were each trimmed with AscI and NotI and were cloned into BssHII-NotI-cut pFastbac1.

Custom synthetic genes encoding the H1 and N1 genes of A/Solomon Islands/3/2006 were obtained from GeneArt (Regensburg, Germany) and were codon-optimized for expression in insect cells. Each fragment contained NotI and KpnI sites at the 5′ and 3′ ends, respectively, for direct cloning into NotI-KpnI-cleaved pFastbac1.

In conducting work involving the use of recombinant DNA the investigators adhered to Guidelines for Research Involving Recombinant DNA Molecules; Notice, Federal Register, Jul. 5, 1994, Volume 59, Number 127.

Purification of VLPs:

Sf9 insect cells were cultured in SF900-II medium and seeded in a 200 ml spinner flask at 5×10⁵ cells per ml. Cells were cultured at 27° C. to a density of 2×10⁶ cells per ml at which time a passage 2 inoculum of VLP-encoding recombinant baculovirus was added at a multiplicity of infection of approximately 0.1 to 1.0. Culture fluids were harvested when cell viability dropped to 20% or below. Medium was clarified of cell debris by centrifugation at 2,000 rpm for 15 minutes after which 32 ml aliquots were layered over 4 ml cushions of 30% sucrose in tris-buffered saline (TBS), pH 7.4. VLPs were centrifuged through the sucrose cushions at 25,000 rpm for 1 hour at 10° C. in a Beckman SW28 rotor (100,000×g). VLPs from a 200 ml culture were resuspended in a total of 6 ml TBS then layered over a single 20-60% discontinuous sucrose gradient and centrifuged at 25,000 rpm for 1 hour at 10° C. Sucrose gradients were fractionated from the bottom into 1.5 ml fractions and analyzed by hemagglutination and neuraminidase assays (see below) and SDS-PAGE and Western blotting. Sucrose gradient purified VLPs were stored at 4° C. in the presence of sucrose and were found to be stable (in terms of HA activity) for at least 6 months. VLPs were centrifuged out of sucrose solutions and resuspended in tris-buffered saline prior to inoculation. VLP vaccines utilized in animal studies reported here were not frozen, however, it was determined that at least one round of H1N1 VLP freezing and thawing could be employed without a measurable loss of HA activity.

Hemagglutination Assays:

Hemagglutination assays were performed as described in the WHO Manual on Animal Influenza Diagnosis and Surveillance using 0.5% chick red blood cells [41].

Neuraminidase Assays:

Neuraminidase activity was detected in VLP preparations and sucrose gradient fractions using the fluorescent substrate 2′-(4-methylumbelliferyl)-α-D-N-acetylneuraminic acid (MUN). Increasing dilutions of VLP-containing samples in PBS (50 μl) were loaded into black flat-bottom 96 well plates and 50 μl PBS was added to each sample well. NA activity was detected by the addition of 50 μl of 35 mM MES, pH 6.5, 4 mM CaCl2, 150 mM NaCl, and 300 μM MUN. Plates were incubated at 37° C. for 1 hour then stopped by the addition of 100 μl per well of 0.14 N NaOH in 83% ethanol. Plates were read in a fluorimeter using excitation and emission wavelengths of 365 nm and 455 nm, respectively. All fluorescence data were corrected for background values obtained from control wells containing the substrate but no VLP. Corrected fluorescence values were then divided by the dilution factor to determine those data points that were in the linear range of the assay.

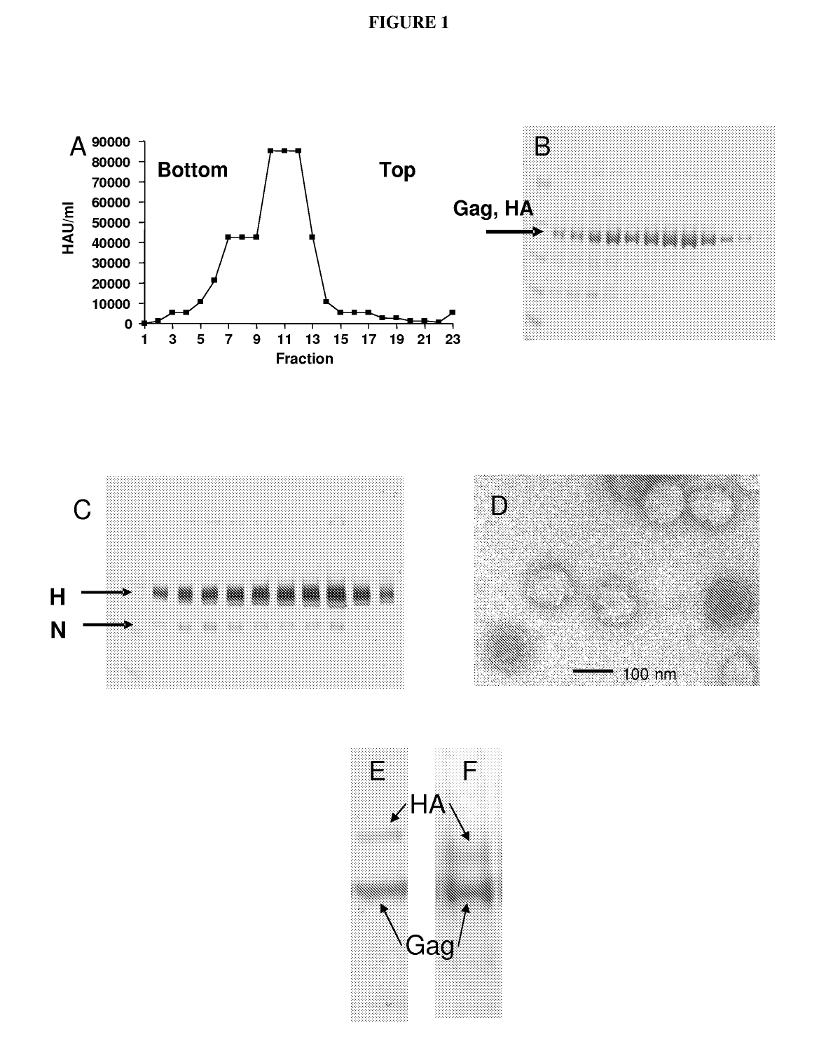

HA-Gag-NA VLPs representing A/PR/8/34 (H1N1) were purified from the medium of Sf9 cells infected with a “triple gene” recombinant baculovirus as described above by centrifugation on discontinuous sucrose density gradients. Analysis of gradient fractions by hemagglutination assay revealed a strong peak of HA activity (FIG. 1A) associated with a prominent visible band in the center of the gradient. This peak also coincided with a prominent 65 kd product on an SDS-PAGE gel consistent with co-migrating Gag and HA (FIG. 1B) and Western blot signals for HA and NA (FIG. 1C). Finally, direct analysis of the peak fractions by electron microscopy showed an abundance of 100 nm spherical particles that were more reminiscent of gamma retroviruses than influenza (FIG. 1D) as would be expected from use of a retroviral Gag protein as the budding engine. While these particles appeared morphologically distinct from influenza virus and influenza matrix-based VLPs it is important to note that hemagglutination activity in pooled peak fractions was determined to be in the range of 2−3×10⁵ HA units per mg total VLP protein which is similar to that observed for gradient purified, egg-grown A/PR/8/34 virus, demonstrating significant incorporation of functional HA spikes into these particles.

The ratio of Gag to HA in PR/8 H1N1 VLPs was determined to be approximately 3-4:1 using a modified PR/8 H1N1 VLP expression vector in which the HA coding sequence was extended at its amino terminus in order to produce a modified HA product that migrated more slowly than Gag (FIG. 1E). The approximate 3-4:1 Gag-to-HA ratio for PR/8 H1N1 VLPs is consistent with other VLPs we produced that represent modern human influenza strains such as A/Wisconsin/67/2005 (H3N2), A/Solomon Islands/3/2006 (H1N1), and B/Malaysia/2506/2004. These latter VLPs contain HA molecules that exhibit a naturally slower mobility than Gag on SDS gels revealing a similar 3-4:1 ratio of Gag-to-HA (FIG. 1F, Solomon Islands H1N1 VLP).

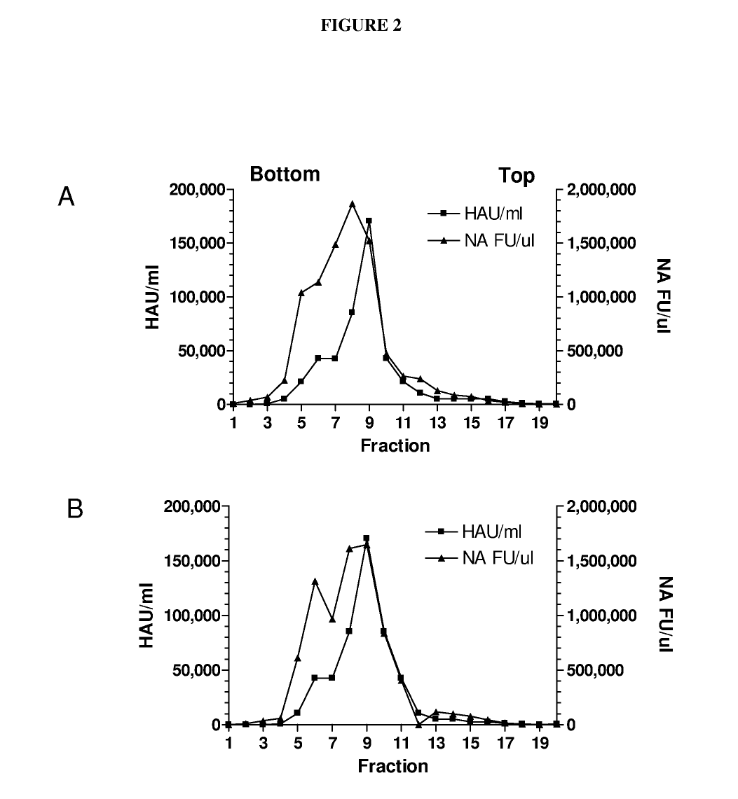

In addition to HA, significant neuraminidase biological activity has also been detected in VLP peaks on sucrose gradients and these data are shown in FIG. 2 for H5N1 VLPs. H5N1 VLPs representing the A/Vietnam/1204/2004 and A/Indonesia/5/2005 strains were produced in a similar manner to VLPs described above with similar yields and hemagglutination specific activities. It is interesting to note that NA activity tends to extend deeper into the gradient overlapping a shoulder of HA activity which is often seen on the heavy side of the HA activity peak. This pattern has been observed for all subtypes tested to date (H1N1, H3N2, and H5N1) and is consistent with the possibility of a continuum of HA:NA ratios in the VLP population (see Example 7).

### Example 2

### H1N1 VLP Immunogenicity and Protection in Mice

Example 2 demonstrates immunization of mice with H1N1 VLPs.

Mouse Immunization and Challenge:

Six-eight week old female Balb/c mice were immunized with VLP formulations in Tris-buffered saline (TBS) containing approximately 0.7 to 1.0 μg HA via intraperitoneal (100 μl) or intramuscular (30 μl) inoculation. In early experiments, 20 monophosphoryl lipid A (detoxified lipid A, Avanti Polar Lipids, Alabaster, Ala.) was added as an adjuvant. Primary and booster immunizations were spaced four weeks apart and blood samples were collected from the lateral facial vein two weeks following each immunization. Immunized and control mice were intranasally challenged with 10 LD50 of egg-grown A/PR/8/34 in 50 μl PBS and were monitored daily for weight loss and morbidity. Animals found moribund or unresponsive to stimuli were euthanized by CO2 inhalation. In conducting research using animals, the investigators adhered to the “Guide for the Care and Use of Laboratory Animals,” prepared by the Committee on Care and Use of Laboratory Animals of the Institute of Laboratory Animal Resources, National Research Council [40].

Hemagglutination Inhibition Assays:

Hemagglutination inhibition (HAI) assays were performed as described in the WHO Manual on Animal Influenza Diagnosis and Surveillance using 0.5% chick red blood cells [41]. For H5N1-specific HAI assays, 1.0% horse red blood cells were also employed and the settling time was increased to 1 hour. H5N1 HAI assays also employed purified H5N1 VLPs as the agglutinating agent in place of infectious virus (see Example 4).

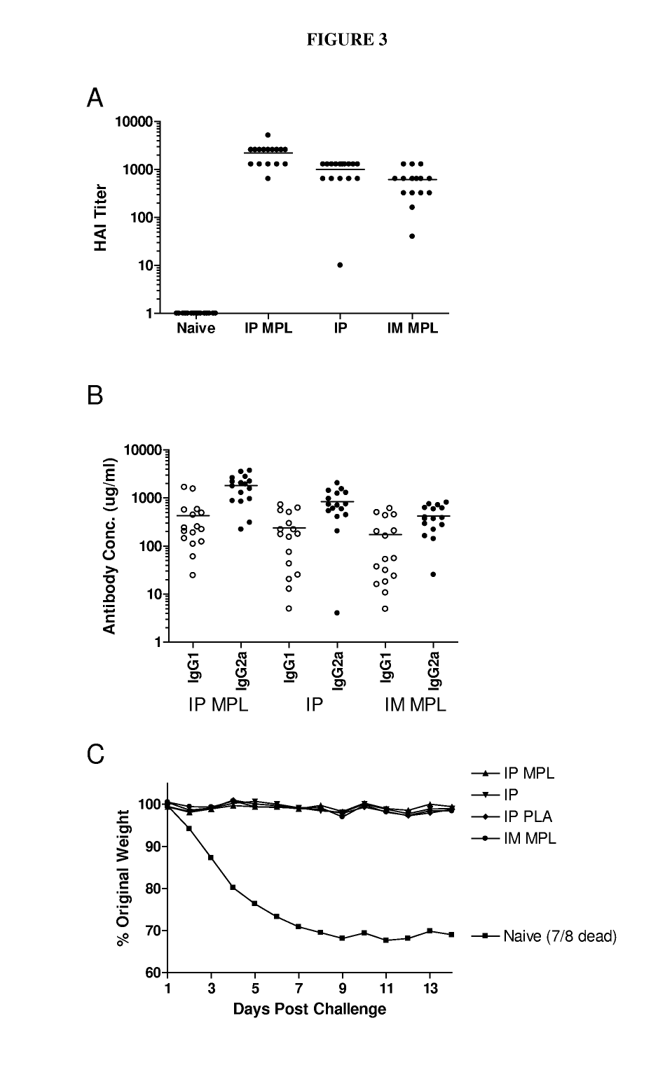

PR/8 H1N1 VLPs in sucrose gradient fractions were pooled on the basis of HA activity and were sedimented out of sucrose and resuspended in PBS for immunization of mice via the intraperitoneal or intramuscular routes with or without monophosphoryl lipid A (MPL) as an adjuvant. Sixteen animals per group received priming and booster immunizations spaced four weeks apart containing approximately 0.7 μg HA per immunization, while 16 naïve animals served as controls. FIG. 3A shows strong A/PR/8/34-specific HAI activity in immunized mice two weeks following the boost and these responses were predominantly of the IgG2a isotype (FIG. 3B). At approximately four weeks post boost each group was challenged with 10 LD50 of mouse-adapted A/PR/8/34 (H1N1). As would be predicted from the H1N1-specific humoral responses, all immunized animals survived the H1N1 challenge with no evidence of morbidity or weight loss while all 8 H1N1-challenged naïve animals showed extensive morbidity and weight loss with 7 of 8 deaths (FIG. 3C). Due to the strength of immune responses in this first trial, the intraperitoneal route of administration was eliminated from further experiments.

### Example 3

### H3N2 and H5N1 VLP Immunogenicity in Mice

Example 3 demonstrates immunization of mice with H3N2 or H5N1 VLPs. Methods of mouse immunization and challenge and of HAI assays were performed as described above in Example 2.

Influenza-Specific Antibody ELISAs:

Egg-grown influenza viruses (A/PR/8/34 (H1N1) or A/Aichi/68 (H3N2)) were centrifuged out of allantoic fluid through a 30% sucrose cushion in TBS in an MLS 50 rotor at 36,000 RPM (100,000×g) for 1 hour at 10° C. Virus pellets were resuspended in PBS and protein content was quantified by BCA assay (Pierce Biotechnology, Rockford, Ill.), adjusted to 5 μg per ml, and used to coat flat-bottom ELISA plates at 100 μl per well overnight at 4° C. Next day, plates were washed with PBS containing 0.05% Tween 20 (PBS-T) and blocked for 30 minutes with StartingBlock(PBS) (Pierce Biotechnology) at 350 μl per well. Serum samples were diluted 1:500 in StartingBlock T20(PBS) (Pierce Biotechnology) and were added to the top row and were serially diluted three-fold down each column and incubated at room temperature for three hours or overnight at 4° C. Plates were washed three times with PBS-T and 100 μl of a 1:1000 diluted goat-anti-mouse IgG-HRP conjugate (Southern Biotech, Birmingham, Ala.) in StartingBlock T20(PBS) was added to each well. Plates were further incubated for 1.5 hours at room temperature, washed three times with PBS-T then 100 μl of ABTS substrate (Pierce Biotechnology) was added to each well. Plates were read at 405 nm after 45 minutes at room temperature. ELISA titers were calculated by taking the reciprocal of the highest serum dilution that yielded an absorbance value 1.75 times the background absorbance value. This cutoff level eliminated any false positives from all naïve serum sample controls.

H5N1-specific ELISAs were similarly performed except that plates were coated with a recombinant H5 Vietnam antigen (Protein Sciences Inc., Meriden, Conn.) or a split H5N1 vaccine formulation at 3 μg per ml (gift of Dr. Sally Mossman, GlaxoSmithKline, Rixensart, Belgium).

IgG1 and IgG2a-specific ELISAs were also performed in a similar manner except that secondary antibodies specific for IgG1 and IgG2a rather than total IgG were employed. For quantification, a series of IgG1 and IgG2a concentration standards were coated onto a series of wells to develop a standard curve for reactivity with the specific secondary antibodies.

Results:

VLPs representing A/Hong Kong/68 (H3N2) were produced as described in Example 1. The immunogenicity of the H3N2 and H5N1 VLPs (along with PR/8 H1N1 VLPs) was evaluated in a mouse immunization trial as shown in Table 1 in which intramuscular priming and booster immunizations containing approximately 1 μg of HA per dose in the presence of MPL adjuvant induced strong HAI activity in all animals. Interestingly, considerable cross-clade HAI activity was observed between the Indonesia and Vietnam H5N1 immunization groups using a horse RBC HAI assay [43] suggesting the potential for inducing significant cross-clade protection against H5N1 challenge.

TABLE 1
HAI activities in mice immunized with H1N1, H3N2, and H5N1 VLPs via intramuscular
inoculation (prime and boost on days 0 and 28, respectively).

| Group | Vaccine | HAI Activity (± standard error) | | | |
|---|---|---|---|---|---|
| n = 16 | (~1 μg HA per dose) | PR/8/34 (H1N1) | HK/68 (H3N2) | Indo 5/05 (H5N1)^{a+} | VN 1203/04 (H5N1)^{b+} |
| 1 | Naïve | <20 | <20 | <20 | <20 |
| 2 | H1N1 VLP | 560 ± 62 | | | |
| 3 | H3N2 VLP | | 680 ± 96 | | |
| 4 | Indo H5N1 VLP^{a} | | | 445 ± 93 (chick) | 540 ± 38 (horse) |
| | | | | 3920 ± 359 (horse) | |
| 5 | VN H5N1 VLP^{b} | | | 701 ± 101 (horse) | 137 ± 27 (chick) |
| | | | | | 861 ± 171 (horse) |

H1N1 and H3N2 assays employed 0.5% standardized chick RBCs. H5N1 assays employed both 0.5% chick and 1.0% horse RBCs.
^{a}A/Indonesia/5/05 (H5N1)
^{b}A/Vietnam/1203/04 (H5N1)
⁺HAI assays performed using H5N1 VLPs in place of live H5N1 virus using chick or horse RBCs as indicated

Table 2 shows the results of ELISA analysis of antibody responses from this same experiment in which strong subtype-specific responses to A/PR/8/34 (H1N1), A/Aichi (H3N2), and a recombinant subunit H5 HA antigen (Vietnam) were observed for the respective vaccines. Importantly, the H5N1 VLPs induced weak ELISA activity against A/PR/8/34 (H1N1) which would be expected by virtue of the shared N1 antigen even though the two N1 antigens are separated by some 70 years of drift with 16.1% amino acid sequence divergence (alignment data not shown). Table 2 also shows the results of a re-immunization experiment in which the H1N1 VLP-immunized mice of group 2 were rested for 8 weeks then re-immunized with H3N2 VLPs (prime and boost) with and without the MPL adjuvant. Importantly, H3N2 responses after 2 prior H1N1 VLP immunizations were equivalent to those induced in naïve mice (group 3). These data show that pre-existing immune responses to antigens shared between different VLP subtypes such as Gag and common insect cell membrane antigens do not abrogate the induction of strong influenza-specific responses when animals are re-immunized with a second VLP subtype. The re-immunization data also show that the MPL adjuvant was contributing little to the observed immune responses since vaccine performance was identical with and without MPL addition. MPL was therefore eliminated from subsequent experiments.

TABLE 2
Endpoint ELISA antibody titers in mice immunized with H1N1, H3N2, and H5N1 VLPs
via intramuscular inoculation (prime and boost on days 0 and 28, respectively).

| Group | Vaccine | Endpoint ELISA Titer (± standard error) | | |
|---|---|---|---|---|
| (n = 16) | (~1 μg HA per dose) | PR/8/34 (H1N1) | A/Aichi/68 (H3N2) | VN (H5N1)^{a} |
| 1 | Naive | BD^{b} | BD | BD |
| 2 | H1N1 VLP | 212,625 ± 30,375 | BD | |
| 3 | H3N2 VLP | BD | 111,375 ± 19,388 | |
| 4 | Indo H5N1 VLP^{c} | 562 ± 124 | | 232,875 ± 34,583 |
| 5 | VN H5N1 VLP^{d} | 652 ± 293 | | 283,500 ± 65,662 |
| 2 | H1N1 VLP then | | 111,375 ± 10,175^{f} | |
| (Re-Immunized) | H3N2 VLP^{e} | | 131,625 ± 35,675^{g} | |

Antibody responses specific for H1N1 and H3N2 viruses were measured using live virus-coated ELISA plates. H5N1 assays employed recombinant Vietnam 1203/04 H5 HA protein-coated ELISA plates.
^{a}Recombinant Vietnam/1203/04 H5 HA employed in ELISA
^{b}Below detection
^{c}A/Indonesia/5/05 (H5N1) VLP
^{d}A/Vietnam 1203/04 (H5N1) VLP
^{e}H1N1 VLP prime and boost followed by H3N2 VLP prime and boost
^{f}with MPL adjuvant during re-immunization
^{g}without MPL adjuvant during re-immunization

### Example 4

### Influenza-Pseudotyped VLPs can Substitute for Live Virus in HAI Assays

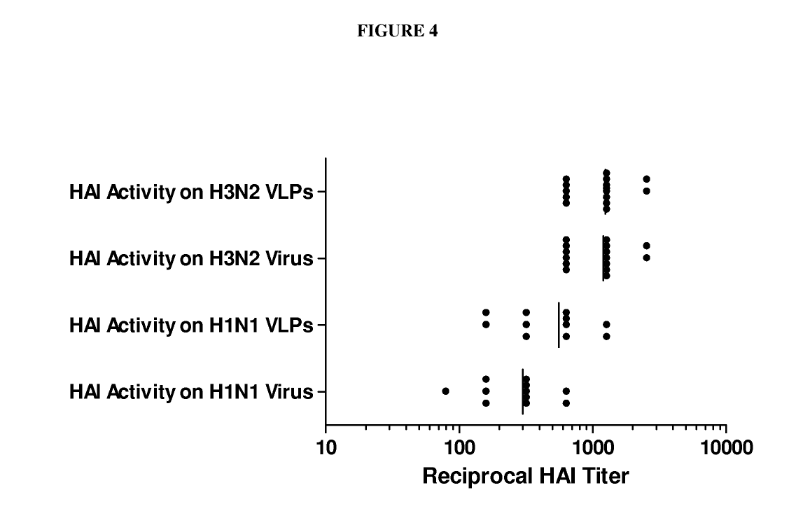

The H5N1 HAI assays shown in Table 1 were performed using H5N1 Indonesia and Vietnam VLPs as a substitute for live virus due to a lack of access to appropriate H5N1 virus strains. HAI assays were performed as described in Example 2. It is important to note that influenza-pseudotyped Gag VLPs can closely mimic the performance of live virus in HAI assays as shown in FIG. 4 for both H1N1 and H3N2 subtypes. In this experiment immune sera from H1N1 and H3N2 VLP-immunized mice were tested for HAI activity using both live virus and corresponding VLPs revealing remarkably similar titers between assay methods. These results not only demonstrate the similar performance between pseudotyped VLPs and virus in HAI assays but provide additional evidence for the ability of pseudotyped VLPs to mimic live influenza viruses in terms of HA activity and densities which is likely important for vaccine performance.

### Example 5

### Highly Pathogenic H5N1 Challenge in Ferrets

Based on the immunogenicity of VLPs in mice we performed a ferret immunization and challenge trial to determine the extent of protection that could be induced against HPAI H5N1 challenge using H5N1 and H1N1 VLP vaccines.

Ferret Vaccination and Challenge:

Thirty-two male ferrets, 8 to 16 weeks of age (Triple F Farms, Sayre, Pa.), serologically negative by hemagglutination inhibition for currently circulating influenza viruses, were vaccinated on days 0 and 28 with VLP vaccine formulations indicated in the text below. Ferret VLP vaccines were formulated in saline without adjuvant and contained approximately 5 μg HA per dose. Blood samples (via anterior vena cava) were collected on days 0, 28, and 42. Body weight measurements were taken daily from a week prior to challenge through study end (day 63). Temperatures were measured via IPTT-300 implantable transponders with a DAS-7000 hand held probe (BioMedic Data Systems, Seafort, Del.). Seven of eight ferrets in each immunization group were randomly selected for challenge. Animals were anesthetized via intramuscular injection with Telezol (16 mg/kg) and intranasally inoculated with 106 TCID50 of A/Vietnam/1203/04 virus in 0.6 ml of PBS. Challenge virus was provided by Dr. Alexander Klimov (CDC, Atlanta, Ga., USA). Ferrets were monitored for changes in body temperature and weight and observed for clinical signs of disease. Any ferret that exhibited neurological dysfunction or became moribund was euthanatized. Animal research was conducted under the supervision of Battelle's Institutional Animal Care and Use Committee in an Association for Assessment and Accreditation of Laboratory Animal Care International-accredited animal facility following the Guide for the Care and Use of Laboratory Animals [40].

Quantification of Virus in Ferret Nasal Washes:

Virus shedding was measured in nasal washes collected on days 3 and 5 post-challenge from anesthetized ferrets. 500 μL of PBS was briskly flushed into each nasal cavity using a 1 mL syringe capped with a flexible catheter for a total of 1 ml of nasal wash. Nasal wash fluids were collected in a sterile specimen cup, transferred to a cryovial and stored at −70° C. until enumeration by TCID50. Virus in nasal washes was quantified by median tissue culture infectious dose on Madin-Darby canine kidney (MDCK) cells. Serial dilutions of virus were plated on cell monolayers in quintuplicate in a 96 well format in EMEM (augmented with 2 mM glutamine, 1 mM sodium pyruvate and 1% penicillin-streptomycin). Monolayers were incubated for 96 hours at 37° C. with 5% CO2. Wells showing cytopathic effects (CPE) were scored as positive and the data were analyzed using the Spearman Karber method [42] and reported as TCID50/mL.

Microneutralization Assay:

Serum neutralizing antibody was measured using a standard microneutralization assay. 300 μL of heat inactivated, serially diluted test sera was mixed with an equal volume of EMEM (augmented with 2 mM glutamine, 1 mM sodium pyruvate and 1% penicillin-streptomycin) containing 600 TCID50 of virus. Following a 1 hour incubation at 37° C., 100 uL of each dilution was plated in quintuplicate on MDCK cells in 96 well format. Following 4 days of incubation at 37° C. with 5% CO2, wells containing CPE were scored as negative. Data were analyzed using the Spearman Karber method and reported as the median neutralizing dose (ND50).

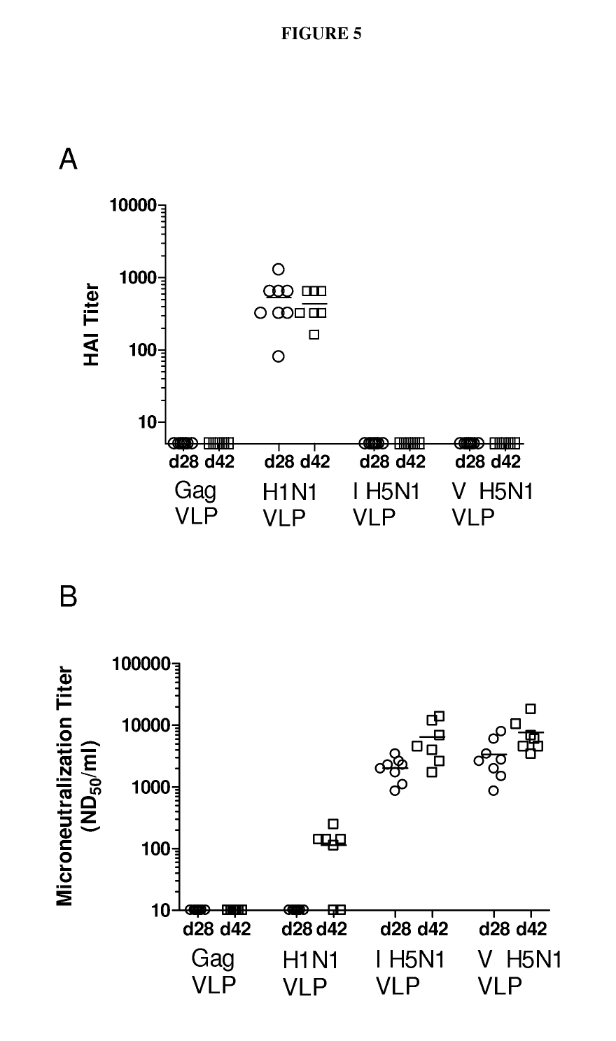

Thirty-two ferrets were randomized into 4 groups (8 animals per group) and immunized with gag-only VLPs, H1N1 (PR/8/34) VLPs, Indonesia H5N1 VLPs, and Vietnam H5N1 VLPs, respectively. VLPs were formulated in saline without MPL adjuvant and primary and booster immunizations were spaced 4 weeks apart. Immunizations contained approximately 5 μg of HA and were administered intramuscularly. After administration of the booster immunization 7 of 8 animals in each group were randomly selected and transferred to and acclimated in the containment facility for subsequent challenge. FIG. 5 shows immune response to H1N1 (FIG. 5A, HAI assay performed as described in Example 2) and Vietnam H5N1 (FIG. 5B, microneutralization assay) in which strong H1N1- and H5N1-specific immune responses were elicited by the respective vaccines following a single immunization. H1N1-specific responses did not further increase after the booster immunization but H5N1 microneutralization titers did increase marginally after the second immunization. Surprisingly, low level H5N1 neutralizing titers were detected in 5 of 7 H1N1-immunized animals following the booster immunization. In addition to H5N1 neutralization activity, strong H5N1 HAI activity specific for both the Indonesia and Vietnam strains was detected in both H5N1 vaccination groups using a horse RBC HAI assay employing Indonesia and Vietnam VLPs. These data are shown in Table 3 in which marked cross-clade HAI activity was observed in both H5N1 vaccine groups.

TABLE 3
H5N1-specific HAI activities in sera from ferrets immunized with
H5N1, H1N1, and Gag-only VLPs via intramuscular inoculation.

HAI Activity (± standard error)

| | Indonesia | Vietnam |
|---|---|---|
| Vaccine | 5/05 (H5N1)^{a} | 1203/04 (H5N1)^{a} |
| Gag (naked VLP) | <10 | 20.0 ± 7.1 |
| H1N1 (PR/8) VLP | 12.1 ± 5.1 | 29.3 ± 21.9 |
| H5N1 Indonesia VLP | 525.7 ± 142.7 | 251.4 ± 75.0 |
| H5N1 Vietnam VLP | 217.1 ± 71.4 | 217.1 ± 71.4 |

^{a}HAI assays performed using H5N1 VLPs and horse RBCs

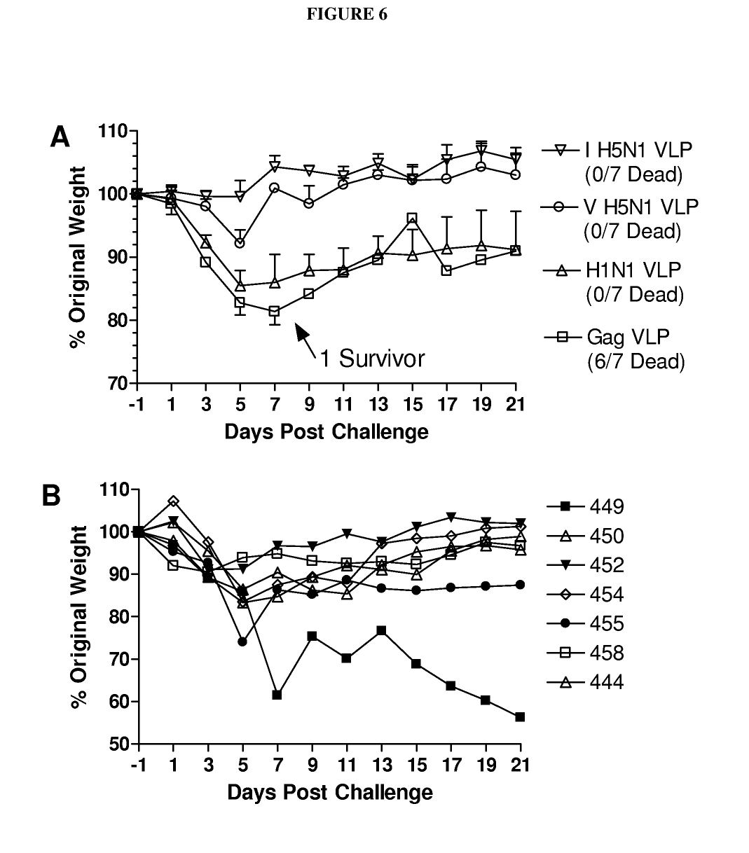

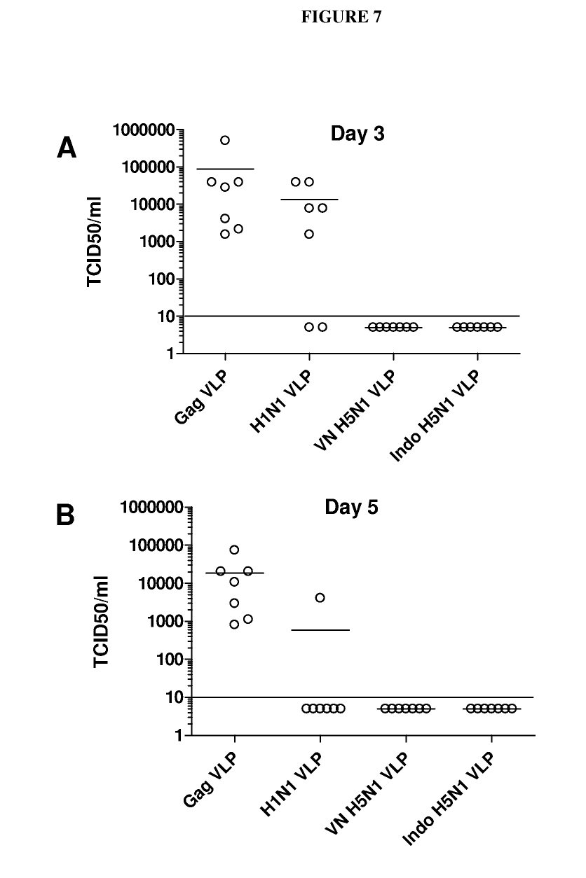

Two weeks following the boost, all ferrets were challenged with 106 TCID50 of HPAI A/Vietnam/1203/04 (H5N1) and weight loss/survival and virus titration data are shown in FIGS. 6 and 7, respectively. As expected from the H5N1 neutralizing titers before challenge all Indonesia and Vietnam H5N1 VLP-vaccinated animals survived the challenge with little indication of morbidity and weight loss while the all Gag VLP vaccinated controls became intensely morbid and 6 of 7 animals required euthanasia (FIG. 6A). This level of morbidity and survival (1 of 7) in the control group was similar to that previously reported for this same dose of A/Vietnam/1203/04 (H5N1) challenge virus [44]. Surprisingly, only moderate morbidity was observed in 6 of 7 H1N1-vaccinated animals and none of the ferrets in this group required euthanasia during the in-life phase of the study. Individual weight loss data for animals in the H1N1 vaccination group are shown in FIG. 6B. One animal in the H1N1 vaccine group (ferret 449) exhibited marked weight loss with time but was not euthanized due to an acceptable alertness and activity level with anorexia being the only persisting symptom. Weight loss in ferret 449 dropped the average weight of the cohort and resulted in higher standard deviations from day 7 post-challenge onward.

Survival, activity and weight loss data are markedly consistent with post-challenge virus isolation data from nasal washes on days 3 and 5 (FIGS. 7A and B) in that H5N1 VLP-vaccinated animals exhibited no significant morbidity following challenge and also exhibited no detectable virus in nasal washes on days 3 and 5 post challenge. In contrast, the extensive morbidity and mortality observed in the Gag VLP-vaccinated ferrets was associated with high levels of virus on both days 3 and 5 post-challenge at levels similar to that previously reported for naïve animals receiving a similar challenge dose [44, 45]. Consistent with the modest morbidity observed in the H1N1 VLP-vaccinated ferrets, post-challenge virus titers were reduced on day 3 relative to the control group and were non-existent in all but one animal on day 5. The one H1N1-vaccinated animal with a nasal wash virus titer on day 5 was the same animal (449) that exhibited greater morbidity and weight loss. The rapid clearance of virus in the remaining 6 animals in the H1N1 groups was associated with the reduced morbidity and more modest weight loss.

The mechanism for partial protection against HPAI H5N1 challenge in the H1N1 VLP-vaccinated ferrets is not known but could be related to the shared N1 antigen between A/PR/8/34 (H1N1) and A/Vietnam/1203/04 (H5N1). The mouse immunogenicity study in Table 2 demonstrates the induction of weak H1N1 ELISA reactivity by both H5N1 VLP vaccines (see Example 3). Similarly, Table 4 shows that the H1N1 VLP vaccine induced weak ELISA reactivity in ferrets against an egg-grown, split H5N1 virus preparation, consistent with the partial protection induced by the H1N1 vaccine. While these ELISA data in no way confirm the role of N1 responses in H1N1-mediated protection against H5N1 challenge, they are consistent with this possibility.

TABLE 4
Endpoint ELISA titers in VLP-immunized ferrets specific
for A/Vietnam/1203/04 (H5N1) split virus.

| | Endpoint ELISA Titer |
|---|---|
| | A/Vietnam/1203/04 (H5N1)^{a} |
| Vaccine | (± standard error) |
| Gag VLP | BD^{b} |
| H1N1 VLP | 529 ± 177 |
| Indonesia H5N1 VLP | 25,071 ± 5456 |
| Vietnam H5N1 VLP | 40,500 ± 14,436 |

^{a}Egg-grown split virus
^{b}Below detection

### Example 6

### Infectious Baculovirus does not Contribute to VLP Immunogenicity

It has been demonstrated that live baculovirus particles exhibit significant innate immune stimulation and adjuvant effects [46-48] and that these properties could play a role in the immunogenicity of insect cell derived products such as VLPs if not removed or inactivated. To investigate this phenomenon we titered baculovirus particles in sucrose density gradients used to purify VLPs representing the virus A/Wisconsin/67/2005 (H3N2) and found baculovirus titers of approximately 2×10⁸ pfu/ml compared to VLP concentrations of approximately 2×10¹² particles per ml in the same fractions. VLP particle quantification was estimated by determining the protein concentration of VLPs and employing a projected VLP molecular weight of 1.2×10⁸ daltons based on 1500 copies of Gag per gamma retrovirus particle and a 4:1 ratio of Gag to HA. Despite the large excess of VLPs over baculovirus, infectious baculovirus was present in VLP preparations and its contribution to immunogenicity was important to investigate.

Inactivation of Baculovirus in VLP Preparations:

Infectious baculovirus in the Wisconsin/67 (H3N2) VLP preparation was inactivated by long wave UV irradiation in the presence of the psoralen derivative 4′-aminomethyl-4,5′,8-trimethylpsoralen hydrochloride (AMT) (Sigma-Aldrich, St. Louis, Mo.) or by treatment with beta-propiolactone (BPL). Pooled sucrose gradient fractions containing VLPs were subjected to either UV or BPL treatment after which VLPs were recovered from the inactivation solutions by centrifugation at 100,000×g through a 30% sucrose cushion in tris-buffered saline, pH 7.4 for 1 hour at 10° C. UV inactivation was carried out by the addition of AMT to 30 μg per ml and 1 ml aliquots of VLP suspension was added to individual wells of a 6-well sterile tissue culture plate. Culture plates (without lids) were placed in a CL-1000 UV crosslinker (UVP, Upland, Calif.) that had been re-configured for long wave radiation and subjected to 2.0 to 2.5 Joules of 365 nm radiation with gentle agitation after every 0.25 Joules. For BPL inactivation, BPL was added to a final concentration of 0.2% and samples were incubated at room temperature for 3 hours.

Three preparations of Wisconsin H3N2 VLPs were subjected to long wave UV/psoralen treatment, beta-propiolactone treatment, or mock inactivation, respectively, and the vaccine preparations were titered for baculovirus infectivity. Both inactivation treatments resulted in reduction of insect cell infectivity to less than 10 infectious baculovirus particles per vaccine dose as determined by endpoint titration on Sf9 cells while the mock inactivated VLP preparation contained between 105 and 106 infectious baculovirus particles per vaccine dose. In addition, UV/psoralen-treated VLPs exhibited no loss of HA activity while BPL-treated VLPs suffered a 16 to 20-reduction in HA activity indicating direct alkylation of the HA antigen and/or disruption of VLP integrity. Intramuscular vaccine doses prepared from the above-treated VLPs contained approximately 1 μg HA for the primary immunization and 0.5 μg HA for the booster immunization. Table 5 shows immunogenicity data for the three vaccine preparations demonstrating no loss of VLP immunogenicity following baculovirus inactivation via UV/psoralen treatment and this is consistent with the retention of full HA activity. These data demonstrate that potential adjuvant properties of infectious baculovirus are not required for the observed pseudotyped VLP immunogenicity. In contrast, the BPL-treated VLPs exhibited greatly reduced immunogenicity, consistent with the marked reduction in HA activity as a result of BPL-treatment.

TABLE 5
Immunogenicity of Wisconsin/67 H3N2 VLPs
in mice following inactivation of baculovirus
with UV/psoralen or BPL treatment.

| | HAI Activity (± standard error) | | | |
|---|---|---|---|---|
| | | Untreated | UV-Treated | BPL-Treated |
| | Naïve | VLP | VLP | VLP |
| Post Prime HAI Titer | <10 | 76 ± 15 | 65 ± 17 | <10 |
| (1.0 μg HA dose) | | | | |
| Post Boost | <10 | 928 ± 121 | 1088 ± 128 | 36 ± 17 |
| (0.5 μg HA dose) | | | | |

Serum samples were collected two weeks following each intramuscular immunization and evaluated for Wisconsin/67 H3N2-specific HAI activity.

### Example 7

### Discussion

We have employed the robust particle budding properties of the MLV Gag gene product for production of chimeric influenza VLPs that exhibit strong immunogenicity and protection in mice and ferrets. Gag-mediated particle budding is dependent upon post-translational myristylation which helps to target these molecules to membrane sites of assembly [31, 32] and evidence is accumulating that Gag-mediated particle budding preferentially occurs through lipid raft domains via association of Gag with caveolin [33]. This process occurs efficiently in both insect and mammalian cells and allows for the incorporation of lipid raft-targeted integral membrane antigens into budded particles without the need for specific interactions between Gag and the cytoplasmic tails of the incorporated antigens. In addition to influenza, this VLP platform should be equally amenable to paramyxovirus antigen incorporation allowing for development of a family of chimeric VLP vaccines for multiple respiratory infectious diseases.

We have built on the earlier findings of influenza HA incorporation into Gag particles by scaling up production of chimeric particles in insect cells, characterizing particles for HA and NA content and biological activities, and launching a number of vaccination studies in mice and ferrets demonstrating strong immunogenicity and protective effects. Chimeric influenza VLPs representing H1N1, H3N2, and H5N1 subtypes are all produced with equal efficiencies in the baculovirus insect cell system and all exhibit hemagglutination specific activities that are similar to those observed with sucrose gradient-purified live influenza viruses in terms of HA units per mg virus or VLP protein. These observations, in addition to the fact that VLPs can be readily substituted for live influenza virus in hemagglutination-inhibition assays, provide compelling evidence that HA antigen display and density on the surface of chimeric VLPs closely mimics that seen with live influenza. We have also demonstrated the production of influenza B virus antigen-containing VLPs showing the flexibility of this system toward viruses other than influenza A (data not shown).

The incorporation of all three genes (HA, Gag, and NA) into one baculovirus vector greatly simplifies VLP production in that product yields are not critically dependent on the multiplicity of infection in insect cells. All chimeric influenza A VLPs produced to date demonstrate similar yields and sucrose gradient banding patterns. These VLPs appear as uniform 100 nm spherical particles by electron microscopy that are strikingly different than the more pleomorphic live influenza or influenza matrix-based VLP particles. This uniformity of VLP size will be important as this technology is further scaled up for production of clinical grade material for human evaluation. Insofar as chimeric VLPs are uniform in size, there appears to be a continuum of relative HA and NA content as shown in FIG. 2 in which both HA and NA activity are measured across a single sucrose gradient. With no expected physical interactions with Gag, the targeting to and sorting within lipid rafts of HA and NA may not be random resulting in VLPs that may show variations in relative HA and NA content. This may have immunological benefits in that HA and NA antigens will be displayed in a variety of contexts on the surface of the VLPs. It is also important to note that the baculovirus gp64 envelope glycoprotein is not a lipid raft protein and would not be expected to accumulate to a significant extent into VLPs [49].

Chimeric VLPs representing H1N1, H3N2, and H5N1 subtypes are strongly immunogenic in the absence of added adjuvants in mice and ferrets leading to vigorous responses to their respective viruses as measured by HAI, neutralization, and ELISA assays. These strong responses resulted in solid protection against homologous challenge in mice and cross-clade H5N1 challenge in ferrets with no evidence for morbidity. While H5N1 cross-clade protection against H5N1 in ferrets is not unprecedented, it is noteworthy that we were unable to detect any virus in post challenge nasal washes in Indonesia H5N1- or Vietnam H5N1-vaccinated animals using a standard 1×10⁶ TCID50 challenge dose of the Vietnam virus. Previous ferret H5N1 vaccine trials have all reported significant quantities of replicating virus in the upper respiratory tract following homosubtypic H5N1 challenge [45, 50, 51].

Understanding the mechanisms of pseudotyped VLP-mediated protection will require further study but a likely contributing factor to the demonstrated vaccine efficacy is the overall strength of immune responses elicited by these vaccines. Strong immunogenicity of VLPs may be due in part to the presence of functional HA spikes on the VLPs that can facilitate efficient binding to essentially any cell, including antigen-presenting cells, leading to enhanced antigen-presentation. Evidence for cell binding capability is the strong HA activity of VLPs observed using chick red cells in both hemagglutination and hemagglutination-inhibition assays. We have also demonstrated strong HA activity toward human RBCs with VLPs of various subtypes (data not shown).

In addition to direct cell binding capability mediated by HA, it has been suggested that the immunogenicity of baculovirus vector-derived VLP vaccines may be augmented by contaminating baculovirus particles since it has been demonstrated that live baculovirus particles can stimulate short term innate immunity and can act as an adjuvant when admixed with other antigens [46-48]. Baculovirus particles band slightly lower in sucrose gradients than VLPs in our hands and quantification of baculovirus by plaque assay and VLPs by protein concentration reveal an abundance of VLPs over baculovirus in VLP preparations by approximately four orders of magnitude. Nevertheless, we did not eliminate baculovirus from the VLP preparations used in most of the experiments described here. However, Table 5 shows the results of a VLP immunogenicity experiment in which contaminating baculovirus particles were inactivated by either long wave UV/psoralen treatment or beta-propiolactone (BPL) treatment. While both methods of inactivation largely target nucleic acids, we observed a marked loss of VLP-associated HA activity following BPL treatment, but no loss of HA activity following UV/psoralen treatment. HAI titers following vaccination revealed that UV/psoralen-treated VLPs suffered no loss of immunogenicity compared to untreated VLPs despite the absence of baculovirus infectivity as determined by titration on insect cells. Thus, contaminating live baculovirus appears to have little impact on immunogenicity associated with these VLPs. Interestingly, BPL-treated VLPs lost most of their immunogenicity which is likely due to direct alkylation of the VLPs as evidenced by a 16 to 20-fold loss of VLP-associated HA activity. Unexpectedly, UV/psoralen inactivation of contaminating baculovirus particles had no effect on VLP immunogenicity; thus, it is an ideal method of inactivation during VLP production.

The issue of low level baculovirus contamination of VLP preparations raises an additional question regarding the extent to which HA-pseudotyped baculovirus particles might contribute to the observed influenza-specific immunogenicity. There are several reasons as to why this is an unlikely possibility. First, baculovirus budded virion (BV) assembly and budding is dependent on membrane-bound baculovirus gp64 [54] which does not target lipid raft domains [49]. Second, the bulk of BV production would be expected to be temporally displaced from the bulk of Gag-VLP assembly since BV production shuts down as the polyhedrin promoter becomes active, driving Gag-HA-NA VLP assembly in the very late stage of the baculovirus infectious cycle. Third, assuming there is some degree of overlap in the relative locations and timing of BV and VLP assembly, the large disparity in the relative amounts of BV and VLPs must be considered. As noted above, our calculations show VLPs in excess of contaminating baculovirus particles by a factor of 104. Such a ratio makes it unlikely that contaminating HA-pseudotyped BV would contribute significantly to vaccine efficacy at the dosage levels employed.

In summary, we describe the production and protective immunogenicity of pseudotyped VLPs consisting of MLV gag and influenza HA and NA from various influenza A subtypes in mice and ferrets. These VLPs are rapidly and consistently produced regardless of strain and demonstrate strong immunogenicity. Our studies of two different methods of inactivating baculovirus particles from VLP preparations yielded the surprising discovery that inactivation by UV/psoralen treatment allows VLPs to maintain their full level of immunogenicity. Experiments to compare the immunogenicity of these VLPs with existing and proposed influenza vaccines as well as to extend the utility of this platform to other respiratory viruses are underway.

### Example 8

### Methods of Virus Inactivation by UV-A, UV-C and Visible Light

In the following example, we describe preferred methods of viral inactivation by UV-A/riboflavin, UV-C irradiation alone and visible light/riboflavin. Virus inactivation may be carried out on any fraction generated during the production of enveloped VLPs including bioreactor offloads, purification starting material following bioreactor harvest by centrifugation or filtration, intermediate purification fractions (continuous flow centrifugation gradient fractions, chromatographic flow-through fractions or chromatographic elution fractions), and/or final purified product.

For virus inactivation studies, enveloped VLP-containing samples will be placed in appropriate, radiation-transparent materials (such as storage bags or tubing) and virus inactivation carried out using devices that may include, but are not limited to: 1) a configuration where single or multiple radiation sources are placed at a set distance, but in any geometry surrounding the sample of interest; 2) a configuration where tubing is attached to any commercially available radiation source to provide a defined sample flow path and sample is passed through the flow path by means of a peristaltic pump or; 3) a configuration where the flow path outlined above is physically integrated as part of a standard unit operation for the purification of enveloped VLPs (e.g. in-line during tangential flow filtration or chromatographic separation).

For pathogen inactivation using UV-A/riboflavin, enveloped VLP samples will be adjusted to riboflavin concentrations of 10-100 μM and then subject to energy doses of 0-50 J/cm² using illumination wavelengths between 265-370 nm. Pathogen inactivation using UV-C will be carried out by treating VLP samples with energy doses of 0-2 J/cm² using a 254 nm illumination wavelength. For pathogen inactivation using visible light/riboflavin, enveloped VLP samples will adjusted to riboflavin concentration of 100-500 μM and then subject to energy doses of 0-500 J/cm² using illumination wavelengths between 400-500 nm. In the above studies, sample conditions such as pH, temperature and the effect of free radical scavengers will be evaluated to optimize inactivation conditions. Representative samples will be exposed to various amounts of radiation and virus inactivation (e.g., baculovirus inactivation) will be assessed by standard infectivity assays. To demonstrate minimal damage to enveloped VLPs, a panel of analytical tests will be performed to show identity, stability and immunogenicity of enveloped VLPs following treatment. Once appropriate virus inactivation conditions have been defined, scale-up studies will be carried out on samples of varying depth to ensure the efficacy of selected virus inactivation methods on levels of material that are more representative of a transferable manufacturing process.

### Example 9

### Methods of Virus Inactivation by Gamma Irradiation

For pathogen inactivation by using gamma irradiation, enveloped VLP samples will be irradiated as both liquid and frozen samples, in the presence and absence of free radical scavengers. Representative samples will be subject to increasing energy doses from 0 to 60 kGy and virus inactivation (e.g., baculovirus inactivation) will be assessed by standard infectivity assays. To demonstrate minimal damage to enveloped VLPs, a panel of analytical tests will performed to show identity, stability and immunogenicity of enveloped VLPs following treatment. Once appropriate virus inactivation conditions have been defined, scale-up studies will be carried out on samples of varying depth to ensure the efficacy of selected virus inactivation methods on levels of material that are more representative of a transferable manufacturing process.

### ADDITIONAL REFERENCES

1. Katz, J. M., W. Lim, C. B. Bridges, T. Rowe, J. Hu-Primmer, X. Lu, R. A. Abernathy, M. Clarke, L. Conn, H. Kwong, M. Lee, G. Au, Y. Y. Ho, K. H. Mak, N. J. Cox, and K. Fukuda. 1999. Antibody response in individuals infected with avian influenza A (H5N1) viruses and detection of anti-H5 antibody among household and social contacts. J Infect Dis 180:1763.
2. Peiris, J. S., W. C. Yu, C. W. Leung, C. Y. Cheung, W. F. Ng, J. M. Nicholls, T. K. Ng, K. H. Chan, S. T. Lai, W. L. Lim, K. Y. Yuen, and Y. Guan. 2004. Re-emergence of fatal human influenza A subtype H5N1 disease. Lancet 363:617.
3. Horimoto, T., N. Fukuda, K. Iwatsuki-Horimoto, Y. Guan, W. Lim, M. Peiris, S. Sugii, T. Odagiri, M. Tashiro, and Y. Kawaoka. 2004. Antigenic differences between H5N1 human influenza viruses isolated in 1997 and 2003. J Vet Med Sci 66:303.
4. Tran, T. H., T. L. Nguyen, T. D. Nguyen, T. S. Luong, P. M. Pham, V. C. Nguyen, T. S. Pham, C. D. Vo, T. Q. Le, T. T. Ngo, B. K. Dao, P. P. Le, T. T. Nguyen, T. L. Hoang, V. T. Cao, T. G. Le, D. T. Nguyen, H. N. Le, K. T. Nguyen, H. S. Le, V. T. Le, D. Christiane, T. T. Tran, J. Menno de, C. Schultsz, P. Cheng, W. Lim, P. Horby, and J. Farrar. 2004. Avian influenza A (H5N1) in 10 patients in Vietnam. N Engl J Med 350:1179.
5. Li, K. S., Y. Guan, J. Wang, G. J. Smith, K. M. Xu, L. Duan, A. P. Rahardjo, P. Puthavathana, C. Buranathai, T. D. Nguyen, A. T. Estoepangestie, A. Chaisingh, P. Auewarakul, H. T. Long, N. T. Hanh, R. J. Webby, L. L. Poon, H. Chen, K. F. Shortridge, K. Y. Yuen, R. G. Webster, and J. S. Peiris. 2004. Genesis of a highly pathogenic and potentially pandemic H5N1 influenza virus in eastern Asia. Nature 430:209.
6. Lipatov, A. S., E. A. Govorkova, R. J. Webby, H. Ozaki, M. Peiris, Y. Guan, L. Poon, and R. G. Webster. 2004. Influenza: emergence and control. J Virol 78:8951.
7. Lipatov, A. S., R. J. Webby, E. A. Govorkova, S. Krauss, and R. G. Webster. 2005. Efficacy of H5 influenza vaccines produced by reverse genetics in a lethal mouse model. J Infect Dis 191:1216.
8. Stephenson, I., K. G. Nicholson, J. M. Wood, M. C. Zambon, and J. M. Katz. 2004. Confronting the avian influenza threat: vaccine development for a potential pandemic. Lancet Infect Dis 4:499.
9. Liu, M., J. M. Wood, T. Ellis, S. Krauss, P. Seiler, C. Johnson, E. Hoffmann, J. Humberd, D. Hulse, Y. Zhang, R. G. Webster, and D. R. Perez. 2003. Preparation of a standardized, efficacious agricultural H5N3 vaccine by reverse genetics. Virology 314:580.
10. Subbarao, K., H. Chen, D. Swayne, L. Mingay, E. Fodor, G. Brownlee, X. Xu, X. Lu, J. Katz, N. Cox, and Y. Matsuoka. 2003. Evaluation of a genetically modified reassortant H5N1 influenza A virus vaccine candidate generated by plasmid-based reverse genetics. Virology 305:192.
11. Webby, R. J., D. R. Perez, J. S. Coleman, Y. Guan, J. H. Knight, E. A. Govorkova, L. R. McClain-Moss, J. S. Peiris, J. E. Rehg, E. I. Tuomanen, and R. G. Webster. 2004. Responsiveness to a pandemic alert: use of reverse genetics for rapid development of influenza vaccines. Lancet 363:1099.
12. Treanor, J. J., B. E. Wilkinson, F. Masseoud, J. Hu-Primmer, R. Battaglia, D. O'Brien, M. Wolff, G. Rabinovich, W. Blackwelder, and J. M. Katz. 2001. Safety and immunogenicity of a recombinant hemagglutinin vaccine for H5 influenza in humans. Vaccine 19:1732.
13. Stephenson, I., R. Bugarini, K. G. Nicholson, A. Podda, J. M. Wood, M. C. Zambon, and J. M. Katz. 2005. Cross-reactivity to highly pathogenic avian influenza H5N1 viruses after vaccination with nonadjuvanted and MF59-adjuvanted influenza A/Duck/Singapore/97 (H5N3) vaccine: a potential priming strategy. J Infect Dis 191:1210.
14. Nicholson, K. G., A. E. Colegate, A. Podda, I. Stephenson, J. Wood, E. Ypma, and M. C. Zambon. 2001. Safety and antigenicity of non-adjuvanted and MF59-adjuvanted influenza A/Duck/Singapore/97 (H5N3) vaccine: a randomised trial of two potential vaccines against H5N1 influenza. Lancet 357:1937.
15. Subbarao, K., B. R. Murphy, and A. S. Fauci. 2006. Development of effective vaccines against pandemic influenza. Immunity 24:5.
16. Kuper, C. F., P. J. Koornstra, D. M. Hameleers, J. Biewenga, B. J. Spit, A. M. Duijvestijn, P. J. van Breda Vriesman, and T. Sminia. 1992. The role of nasopharyngeal lymphoid tissue. Immunol Today 13:219.
17. Liang, B., L. Hyland, and S. Hou. 2001. Nasal-associated lymphoid tissue is a site of long-term virus-specific antibody production following respiratory virus infection of mice. J Virol 75:5416.
18. Zuercher, A. W., S. E. Coffin, M. C. Thurnheer, P. Fundova, and J. J. Cebra. 2002. Nasal-associated lymphoid tissue is a mucosal inductive site for virus-specific humoral and cellular immune responses. J Immunol 168:1796.
19. Brandtzaeg, P. 1989. Overview of the mucosal immune system. Curr Top Microbiol Immunol 146:13.
20. Takada, A., S. Matsushita, A. Ninomiya, Y. Kawaoka, and H. Kida. 2003. Intranasal immunization with formalin-inactivated virus vaccine induces a broad spectrum of heterosubtypic immunity against influenza A virus infection in mice. Vaccine 21:3212.
21. Tamura, S. I., H. Asanuma, Y. Ito, Y. Hirabayashi, Y. Suzuki, T. Nagamine, C. Aizawa, T. Kurata, and A. Oya. 1992. Superior cross-protective effect of nasal vaccination to subcutaneous inoculation with influenza hemagglutinin vaccine. Eur J Immunol 22:477.
22. Tumpey, T. M., M. Renshaw, J. D. Clements, and J. M. Katz. 2001. Mucosal delivery of inactivated influenza vaccine induces B-cell-dependent heterosubtypic cross-protection against lethal influenza A H5N1 virus infection. J Virol 75:5141.
23. Kang, S. M., L. Guo, Q. Yao, I. Skountzou, and R. W. Compans. 2004. Intranasal immunization with inactivated influenza virus enhances immune responses to coadministered simian-human immunodeficiency virus-like particle antigens. J Virol 78:9624.
24. Guo, L., X. Lu, S. M. Kang, C. Chen, R. W. Compans, and Q. Yao. 2003. Enhancement of mucosal immune responses by chimeric influenza HA/SHIV virus-like particles. Virology 313:502.
25. Yao, Q., R. Zhang, L. Guo, M. Li, and C. Chen. 2004. Th cell-independent immune responses to chimeric hemagglutinin/simian human immunodeficiency virus-like particles vaccine. J Immunol 173:1951.
26. Latham, T., and J. M. Galarza. 2001. Formation of wild-type and chimeric influenza virus-like particles following simultaneous expression of only four structural proteins. J Virol 75:6154.
27. Galarza, J. M., T. Latham, and A. Cupo. 2005. Virus-like particle vaccine conferred complete protection against a lethal influenza virus challenge. Viral Immunol 18:365.
28. Fromantin, C., B. Jamot, J. Cohen, L. Piroth, P. Pothier, and E. Kohli. 2001. Rotavirus 2/6 virus-like particles administered intranasally in mice, with or without the mucosal adjuvants cholera toxin and Escherichia coli heat-labile toxin, induce a Th1/Th2-like immune response. Virol 75:11010.
29. Harrington, P. R., B. Yount, R. E. Johnston, N. Davis, C. Moe, and R. S. Baric. 2002. Systemic, mucosal, and heterotypic immune induction in mice inoculated with Venezuelan equine encephalitis replicons expressing Norwalk virus-like particles. J Virol 76:730.
30. Shi, W., J. Liu, Y. Huang, and L. Qiao. 2001. Papillomavirus pseudovirus: a novel vaccine to induce mucosal and systemic cytotoxic T-lymphocyte responses. J Virol 75:10139.
31. Han, M. G., S. Cheetham, M. Azevedo, C. Thomas, and L. J. Saif. 2006. Immune responses to bovine norovirus-like particles with various adjuvants and analysis of protection in gnotobiotic calves. Vaccine 24:317.
32. Illum, L. 1998. Chitosan and its use as a pharmaceutical excipient. Pharm Res 15:1326.
33. Illum, L., I. Jabbal-Gill, M. Hinchcliffe, A. N. Fisher, and S. S. Davis. 2001. Chitosan as a novel nasal delivery system for vaccines. Adv Drug Deliv Rev 51:81.
34. Soane, R. J., M. Hinchcliffe, S. S. Davis, and L. Illum. 2001. Clearance characteristics of chitosan based formulations in the sheep nasal cavity. Int J Pharm 217:183.
35. Baudner, B. C., M. M. Giuliani, J. C. Verhoef, R. Rappuoli, H. E. Junginger, and G. D. Giudice. 2003. The concomitant use of the LTK63 mucosal adjuvant and of chitosan-based delivery system enhances the immunogenicity and efficacy of intranasally administered vaccines. Vaccine 21:3837.
36. Fujihashi, K., T. Koga, F. W. van Ginkel, Y. Hagiwara, and J. R. McGhee. 2002. A dilemma for mucosal vaccination: efficacy versus toxicity using enterotoxin-based adjuvants. Vaccine 20:2431.
37. Mutsch, M., W. Zhou, P. Rhodes, M. Bopp, R. T. Chen, T. Linder, C. Spyr, and R. Steffen. 2004. Use of the inactivated intranasal influenza vaccine and the risk of Bell's palsy in Switzerland. N Engl J Med 350:896.
38. Baldridge, J. R., Y. Yorgensen, J. R. Ward, and J. T. Ulrich. 2000. Monophosphoryl lipid A enhances mucosal and systemic immunity to vaccine antigens following intranasal administration. Vaccine 18:2416.
39. Baldrick, P., D. Richardson, G. Elliott, and A. W. Wheeler. 2002. Safety evaluation of monophosphoryl lipid A (MPL): an immunostimulatory adjuvant. Regul Toxicol Pharmacol 35:398.
40. Baldridge, J. R., P. McGowan, J. T. Evans, C. Cluff, S. Mossman, D. Johnson, and D. Persing. 2004. Taking a Toll on human disease: Toll-like receptor 4 agonists as vaccine adjuvants and monotherapeutic agents. Expert Opin Biol Ther 4:1129.
41. Baldridge, J. GlaxoSmithKline, Personal Communication.
42. Huang, J., R. J. Garmise, T. M. Crowder, K. Mar, C. R. Hwang, A. J. Hickey, J. A. Mikszta, and V. J. Sullivan. 2004. A novel dry powder influenza vaccine and intranasal delivery technology: induction of systemic and mucosal immune responses in rats. Vaccine 23:794.
43. Mills, K. H., C. Cosgrove, E. A. McNeela, A. Sexton, R. Giemza, I. Jabbal-Gill, A. Church, W. Lin, L. Illum, A. Podda, R. Rappuoli, M. Pizza, G. E. Griffin, and D. J. Lewis. 2003. Protective levels of diphtheria-neutralizing antibody induced in healthy volunteers by unilateral priming-boosting intranasal immunization associated with restricted ipsilateral mucosal secretory immunoglobulin a. Infect Immun 71:726.
44. Wimer-Mackin, S., M. Hinchcliffe, C. R. Petrie, S. J. Warwood, W. T. Tino, M. S. Williams, J. P. Stenz, A. Cheff, and C. Richardson. 2006. An intranasal vaccine targeting both the Bacillus anthracis toxin and bacterium provides protection against aerosol spore challenge in rabbits. Vaccine in press.
45. Noad, R., and P. Roy. 2003. Virus-like particles as immunogens. Trends Microbiol 11:438.
46. Yao, Q., V. Vuong, M. Li, and R. W. Compans. 2002. Intranasal immunization with SIV virus-like particles (VLPs) elicits systemic and mucosal immunity. Vaccine 20:2537.
47. Pushko, P., T. M. Tumpey, F. Bu, J. Knell, R. Robinson, and G. Smith. 2005. Influenza virus-like particles comprised of the HA, NA, and M1 proteins of H9N2 influenza virus induce protective immune responses in BALB/c mice. Vaccine 23:5751.
48. Baumert, T. F., S. Ito, D. T. Wong, and T. J. Liang. 1998. Hepatitis C virus structural proteins assemble into viruslike particles in insect cells. J Virol 72:3827.
49. Yao, Q., Z. Bu, A. Vzorov, C. Yang, and R. W. Compans. 2003. Virus-like particle and DNA-based candidate AIDS vaccines. Vaccine 21:638.
50. Tacket, C. O., M. B. Sztein, G. A. Losonsky, S. S. Wasserman, and M. K. Estes. 2003. Humoral, mucosal, and cellular immune responses to oral Norwalk virus-like particles in volunteers. Clin Immunol 108:241.
51. Gomez-Puertas, P., C. Albo, E. Perez-Pastrana, A. Vivo, and A. Portela. 2000. Influenza virus matrix protein is the major driving force in virus budding. J Virol 74:11538.
52. Gheysen, D., E. Jacobs, F. de Foresta, C. Thiriart, M. Francotte, D. Thines, and M. De Wilde. 1989. Assembly and release of HIV-1 precursor Pr55gag virus-like particles from recombinant baculovirus-infected insect cells. Cell 59:103.
53. Johnson, M. C., H. M. Scobie, and V. M. Vogt. 2001. PR domain of rous sarcoma virus Gag causes an assembly/budding defect in insect cells. J Virol 75:4407.
54. Kakker, N. K., M. V. Mikhailov, M. V. Nermut, A. Burny, and P. Roy. 1999. Bovine leukemia virus Gag particle assembly in insect cells: formation of chimeric particles by domain-switched leukemia/lentivirus Gag polyprotein. Virology 265:308.
55. Luo, L., Y. Li, and C. Y. Kang. 1990. Expression of gag precursor protein and secretion of virus-like gag particles of HIV-2 from recombinant baculovirus-infected insect cells. Virology 179:874.
56. Morikawa, S., T. F. Booth, and D. H. Bishop. 1991. Analyses of the requirements for the synthesis of virus-like particles by feline immunodeficiency virus gag using baculovirus vectors. Virology 183:288.
57. Takahashi, R. H., K. Nagashima, T. Kurata, and H. Takahashi. 1999. Analysis of human lymphotropic T-cell virus type II-like particle production by recombinant baculovirus-infected insect cells. Virology 256:371.
58. Yamshchikov, G. V., G. D. Ritter, M. Vey, and R. W. Compans. 1995. Assembly of SIV virus-like particles containing envelope proteins using a baculovirus expression system. Virology 214:50.
59. Weldon, R. A., Jr., C. R. Erdie, M. G. Oliver, and J. W. Wills. 1990. Incorporation of chimeric gag protein into retroviral particles. J Virol 64:4169.
60. Andrawiss, M., Y. Takeuchi, L. Hewlett, and M. Collins. 2003. Murine leukemia virus particle assembly quantitated by fluorescence microscopy: role of Gag-Gag interactions and membrane association. J Virol 77:11651.
61. Leser, G. P., and R. A. Lamb. 2005. Influenza virus assembly and budding in raft-derived microdomains: a quantitative analysis of the surface distribution of HA, NA and M2 proteins. Virology 342:215.
62. Takeda, M., G. P. Leser, C. J. Russell, and R. A. Lamb. 2003. Influenza virus hemagglutinin concentrates in lipid raft microdomains for efficient viral fusion. Proc Natl Acad Sci USA 100:14610.
63. Campbell, S. M., S. M. Crowe, and J. Mak. 2001. Lipid rafts and HIV-1: from viral entry to assembly of progeny virions. J Clin Virol 22:217.
64. Sandrin, V., and F. L. Cosset. 2006. Intracellular versus cell surface assembly of retroviral pseudotypes is determined by the cellular localization of the viral glycoprotein, its capacity to interact with Gag, and the expression of the Nef protein. J Biol Chem 281:528.
65. Salazar-Gonzalez, R. M., and S. J. McSorley. 2005. Salmonella flagellin, a microbial target of the innate and adaptive immune system. Immunol Lett 101:117.
66. Cuadros, C., F. J. Lopez-Hernandez, A. L. Dominguez, M. McClelland, and J. Lustgarten. 2004. Flagellin fusion proteins as adjuvants or vaccines induce specific immune responses. Infect Immun 72:2810.
67. Didierlaurent, A., I. Ferrero, L. A. Otten, B. Dubois, M. Reinhardt, H. Carlsen, R. Blomhoff, S. Akira, J. P. Kraehenbuhl, and J. C. Sirard. 2004. Flagellin promotes myeloid differentiation factor 88-dependent development of Th2-type response. J Immunol 172:6922.
68. Tsujimoto, H., T. Uchida, P. A. Efron, P. O, Scumpia, A. Verma, T. Matsumoto, S. K. Tschoeke, R. F. Ungaro, S. Ono, S. Seki, M. J. Clare-Salzler, H. V. Baker, H. Mochizuki, R. Ramphal, and L. L. Moldawer. 2005. Flagellin enhances NK cell proliferation and activation directly and through dendritic cell-NK cell interactions. J Leukoc Biol 78:888.
69. Hayashi, F., T. K. Means, and A. D. Luster. 2003. Toll-like receptors stimulate human neutrophil function. Blood 102:2660.
70. Hayashi, F., K. D. Smith, A. Ozinsky, T. R. Hawn, E. C. Yi, D. R. Goodlett, J. K. Eng, S. Akira, D. M. Underhill, and A. Aderem. 2001. The innate immune response to bacterial flagellin is mediated by Toll-like receptor 5. Nature 410:1099.
71. Gewirtz, A. T., P. O, Simon, Jr., C. K. Schmitt, L. J. Taylor, C. H. Hagedorn, A. D. O'Brien, A. S, Neish, and J. L. Madara. 2001. Salmonella typhimurium translocates flagellin across intestinal epithelia, inducing a proinflammatory response. J Clin Invest 107:99.
72. Means, T. K., F. Hayashi, K. D. Smith, A. Aderem, and A. D. Luster. 2003. The Toll-like receptor 5 stimulus bacterial flagellin induces maturation and chemokine production in human dendritic cells. J Immunol 170:5165.
73. Smith, K. D., E. Andersen-Nissen, F. Hayashi, K. Strobe, M. A. Bergman, S. L. Barrett, B. T. Cookson, and A. Aderem. 2003. Toll-like receptor 5 recognizes a conserved site on flagellin required for protofilament formation and bacterial motility. Nat Immunol 4:1247.
74. Honko, A. N., N. Sriranganathan, C. J. Lees, and S. B. Mizel. 2006. Flagellin is an effective adjuvant for immunization against lethal respiratory challenge with Yersinia pestis. Infect Immun 74:1113.
75. Jeon, S. H., T. Ben-Yedidia, and R. Arnon. 2002. Intranasal immunization with synthetic recombinant vaccine containing multiple epitopes of influenza virus. Vaccine 20:2772.
76. Levi, R., and R. Arnon. 1996. Synthetic recombinant influenza vaccine induces efficient long-term immunity and cross-strain protection. Vaccine 14:85.
77. Lee, S. E., S. Y. Kim, B. C. Jeong, Y. R. Kim, S. J. Bae, O, S. Ahn, J. J. Lee, H. C. Song, J. M. Kim, H. E. Choy, S. S. Chung, M. N. Kweon, and J. H. Rhee. 2006. A bacterial flagellin, Vibrio vulnificus FlaB, has a strong mucosal adjuvant activity to induce protective immunity. Infect Immun 74:694.
78. Applequist, S. E., E. Rollman, M. D. Wareing, M. Liden, B. Rozell, J. Hinkula, and H. G. Ljunggren. 2005. Activation of innate immunity, inflammation, and potentiation of DNA vaccination through mammalian expression of the TLR5 agonist flagellin. J Immunol 175:3882.
79. Ramos, H. C., M. Rumbo, and J. C. Sirard. 2004. Bacterial flagellins: mediators of pathogenicity and host immune responses in mucosa. Trends Microbiol 12:509.
80. Holsinger, L. J., and R. A. Lamb. 1991. Influenza virus M2 integral membrane protein is a homotetramer stabilized by formation of disulfide bonds. Virology 183:32.
81. Lamb, R. A., S. L. Zebedee, and C. D. Richardson. 1985. Influenza virus M2 protein is an integral membrane protein expressed on the infected-cell surface. Cell 40:627.
82. Holsinger, L. J., D. Nichani, L. H. Pinto, and R. A. Lamb. 1994. Influenza A virus M2 ion channel protein: a structure-function analysis. J Virol 68:1551.
83. Takeda, M., A. Pekosz, K. Shuck, L. H. Pinto, and R. A. Lamb. 2002. Influenza a virus M2 ion channel activity is essential for efficient replication in tissue culture. J Virol 76:1391.
84. Frace, A. M., A. I. Klimov, T. Rowe, R. A. Black, and J. M. Katz. 1999. Modified M2 proteins produce heterotypic immunity against influenza A virus. Vaccine 17:2237.
85. Neirynck, S., T. Deroo, X. Saelens, P. Vanlandschoot, W. M. Jou, and W. Fiers. 1999. A universal influenza A vaccine based on the extracellular domain of the M2 protein. Nat Med 5:1157.
86. Slepushkin, V. A., J. M. Katz, R. A. Black, W. C. Gamble, P. A. Rota, and N. J. Cox. 1995. Protection of mice against influenza A virus challenge by vaccination with baculovirus-expressed M2 protein. Vaccine 13:1399.
87. Treanor, J. J., E. L. Tierney, S. L. Zebedee, R. A. Lamb, and B. R. Murphy. 1990. Passively transferred monoclonal antibody to the M2 protein inhibits influenza A virus replication in mice. J Virol 64:1375.
88. De Filette, M., W. Min Jou, A. Birkett, K. Lyons, B. Schultz, A. Tonkyro, S. Resch, and W. Fiers. 2005. Universal influenza A vaccine: optimization of M2-based constructs. Virology 337:149.
89. De Filette, M., A. Ramne, A. Birkett, N. Lycke, B. Lowenadler, W. Min Jou, X. Saelens, and W. Fiers. 2006. The universal influenza vaccine M2e-HBc administered intranasally in combination with the adjuvant CTA1-DD provides complete protection. Vaccine 24:544.
90. Fiers, W., M. De Filette, A. Birkett, S, Neirynck, and W. Min Jou. 2004. A “universal” human influenza A vaccine. Virus Res 103:173.
91. Liu, W., Z. Peng, Z. Liu, Y. Lu, J. Ding, and Y. H. Chen. 2004. High epitope density in a single recombinant protein molecule of the extracellular domain of influenza A virus M2 protein significantly enhances protective immunity. Vaccine 23:366.
92. Fan, J., X. Liang, M. S. Horton, H. C. Perry, M. P. Citron, G. J. Heidecker, T. M. Fu, J. Joyce, C. T. Przysiecki, P. M. Keller, V. M. Garsky, R. Ionescu, Y. Rippeon, L. Shi, M. A. Chastain, J. H. Condra, M. E. Davies, J. Liao, E. A. Emini, and J. W. Shiver. 2004. Preclinical study of influenza virus A M2 peptide conjugate vaccines in mice, ferrets, and rhesus monkeys. Vaccine 22:2993.
93. Ionescu, R. M., C. T. Przysiecki, X. Liang, V. M. Garsky, J. Fan, B. Wang, R. Troutman, Y. Rippeon, E. Flanagan, J. Shiver, and L. Shi. 2006. Pharmaceutical and immunological evaluation of human papillomavirus viruslike particle as an antigen carrier. J Pharm Sci 95:70.
94. Hatziioannou, T., E. Delahaye, F. Martin, S. J. Russell, and F. L. Cosset. 1999. Retroviral display of functional binding domains fused to the amino terminus of influenza hemagglutinin. Hum Gene Ther 10:1533.
95. Li, Z. N., S, N. Mueller, L. Ye, Z. Bu, C. Yang, R. Ahmed, and D. A. Steinhauer. 2005. Chimeric influenza virus hemagglutinin proteins containing large domains of the Bacillus anthracis protective antigen: protein characterization, incorporation into infectious influenza viruses, and antigenicity. J Virol 79:10003.
96. Haynes, J. R., S. X. Cao, B. Rovinski, C. Sia, O. James, G. A. Dekaban, and M. H. Klein. 1991. Production of immunogenic HIV-1 viruslike particles in stably engineered monkey cell lines. AIDS Res Hum Retroviruses 7:17.
97. Rovinski, B., J. R. Haynes, S. X. Cao, O. James, C. Sia, S. Zolla-Pazner, T. J. Matthews, and M. H. Klein. 1992. Expression and characterization of genetically engineered human immunodeficiency virus-like particles containing modified envelope glycoproteins: implications for development of a cross-protective AIDS vaccine. J Virol 66:4003.
98. Fynan, E. F., R. G. Webster, D. H. Fuller, J. R. Haynes, J. C. Santoro, and H. L. Robinson. 1995. DNA vaccines: a novel approach to immunization. Int J Immunopharmacol 17:79.
99. Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proc Natl Acad Sci USA 90:11478.
100. Kodihalli, S., J. R. Haynes, H. L. Robinson, and R. G. Webster. 1997. Cross-protection among lethal H5N2 influenza viruses induced by DNA vaccine to the hemagglutinin. J Virol 71:3391.
101. Robinson, H. L., S. Lu, D. M. Feltquate, C. T. Tones, J. Richmond, C. M. Boyle, M. J. Morin, J. C. Santoro, R. G. Webster, D. Montefiori, Y. Yasutomi, N. L. Letvin, K. Manson, M. Wyand, and J. R. Haynes. 1996. DNA vaccines. AIDS Res Hum Retroviruses 12:455.
102. Drape, R. J., M. D. Macklin, L. J. Barr, S. Jones, J. R. Haynes, and H. J. Dean. 2006. Epidermal DNA vaccine for influenza is immunogenic in humans. Vaccine in press.
103. Kretzchmar, E., R. Geyer, and H. D. Klenk. 1994. Baculovirus infection does not alter N-glycosylation in Spodoptera frugiperda cells. Biol Chem Hoppe Seyler 375:23.
104. Lu, D., and A. J. Hickey. 2005. Liposomal dry powders as aerosols for pulmonary delivery of proteins. AAPS PharmSciTech 6:E641.
105. Cowdery, S., M. Frey, S. Orlowski, and A. Gray. 1976. Stability characteristics of freeze-dried human live virus vaccines. Dev Biol Stand 36:297.
106. Peetermans, J., G. Colinet, A. Bouillet, E. D'Hondt, and J. Stephenne. 1976. Stability of live, freeze-dried virus vaccines. Dev Biol Stand 36:291.
107. Yannarell, D. A., K. M. Goldberg, and R. N. Hjorth. 2002. Stabilizing cold-adapted influenza virus vaccine under various storage conditions. J Virol Methods 102:15.
108. Sampson, H. A., J. Bernhisel-Broadbent, E. Yang, and S. M. Scanlon. 1991. Safety of casein hydrolysate formula in children with cow milk allergy. J Pediatr 118:520.
109. Gambaryan, A. A. Tuzikov, G. Pazynina, N. Bovin, A. Balish, and A. Klimov. 2005. Evolution of the receptor binding phenotype of influenza A (H5) viruses. Virology (electronic publication ahead of print version).
110. Suzuki, Y, 2005. Sialobiology of Influenza: Molecular Mechanism of Host Range Variation of Influenza Viruses. Biological and Pharmaceutical Bulletin 28:399-408.
111. Latham, T., and J. M. Galarza. 2001. Formation of wild-type and chimeric influenza virus-like particles following simultaneous expression of only four structural proteins. J Virol. 75(13):6154-65.
112. Galarza, J. M., T. Latham, and A. Cupo. 2005. Virus-like particle (VLP) vaccine conferred complete protection against a lethal influenza virus challenge. Viral Immunol. 18(1):244-51.

1. A method for isolating an enveloped virus-based virus-like particle preparation substantially free of infectious agents comprising:
(a) separating the enveloped virus-based virus-like particle preparation from host cells used to generate the enveloped virus-based virus-like particle preparation or from a component of the host cells; and
(b) applying a sufficient dose of electromagnetic radiation to the enveloped virus-based virus-like particle preparation to inactivate substantially all of infectious agents in the preparation,
wherein the enveloped virus-based virus-like particle preparation after step (b) has substantially the same immunogenicity as the enveloped virus-based virus-like particle preparation prior to step (b).

2. The method of claim 1, wherein the separating step (a) comprises at least one chromatographic step.

3. The method of claim 2, wherein said at least one chromatographic step is selected from the group consisting of ion-exchange, affinity, hydrophobic interaction, mixed mode, reversed phase, and size exclusion

4. The method of claim 3, wherein the separating step (a) comprises at least one filtration step, a centrifugation step, or both.

5. The method of claim 4, wherein said at least one filtration step is normal flow filtration, ultrafiltration or tangential flow filtration.

6. (canceled)

7. The method of claim 1, wherein the electromagnetic radiation is selected from the group consisting of visible, x-ray, ultraviolet and gamma radiation.

8. The method of claim 7, wherein the ultraviolet radiation is selected from the group consisting of UV-A, UV-B and UV-C.

9. The method of claim 7, wherein the ultraviolet radiation has a wavelength between 320 nm and 400 nm.

10. (canceled)

11. The method of claim 1, wherein said host cells are insect cells and said insect cells are infected with a baculovirus expression vector that expresses at least one component of the enveloped virus-based virus-like particle.

12. (canceled)

13. The method of claim 11, wherein the dose of electromagnetic radiation is sufficient to inactivate baculovirus in the enveloped virus-based virus-like particle preparation.

14. (canceled)

15. The method of claim 1, further comprising adding a DNA intercalating compound to the enveloped virus-based virus-like particle preparation prior to the applying step (b).

16. The method of claim 15, wherein the DNA intercalating compound is photoreactive.

17-20. (canceled)

21. The method of claim 1, wherein the enveloped virus-based virus-like particle is produced in the host cell prior to the separating step (a) by (i) providing one or more expression vectors, which expresses a respiratory syncytial virus M polypeptide and a respiratory syncytial virus F polypeptide; (ii) introducing said one or more expression vectors into a cell; and (iii) expressing said respiratory syncytial virus M polypeptide and said respiratory syncytial virus F polypeptide to produce said virus-like particle.

22. (canceled)

23. The method of claim 1, wherein the enveloped virus-based virus-like particle is produced in the host cell prior to the separating step (a) by (i) providing one or more expression vectors, which together express a gag polypeptide and an influenza hemagglutinin polypeptide; (ii) introducing said one or more expression vectors into a cell; and (iii) expressing said gag polypeptide and said influenza hemagglutinin polypeptide to produce said virus-like particle.

24. The method of claim 1, wherein the enveloped virus-based virus-like particle is produced in the host cell prior to the separating step (a) by (i) providing one or more expression vectors, which together which express an influenza M1 polypeptide and an hemagglutinin polypeptide; (ii) introducing said one or more expression vectors into a cell; and (iii) expressing said influenza M1 polypeptide and said hemagglutinin polypeptide to produce said virus-like particle.

25-35. (canceled)

36. The method of claim 1, wherein the enveloped virus-based virus-like particle preparation after step (b) has at least fifty percent of the immunogenicity of the enveloped virus-based virus-like particle preparation prior to step (b).

37-43. (canceled)

44. An enveloped virus-based virus-like particle preparation comprising enveloped virus-based virus-like particles that are substantially free of infectious agents wherein the enveloped virus-based virus-like particles have substantially the same immunogenicity as enveloped virus-based virus-like particles that are not substantially free of infectious agents.

45. The enveloped virus based virus like particle preparation of claim 44 wherein the enveloped virus-based virus-like particles further comprise insect or mammalian glycosylation.

46. (canceled)

47. The enveloped virus based virus like particle preparation of claim 44 wherein the enveloped virus-based virus-like particles further lack one or more defects selected from: covalently linked photochemical agents, UV- or gamma-irradiation induced changes in the tertiary or the quaternary structure of protein subunits, gamma irradiation induced chemical bond cleavage, or UV- or gamma-irradiation induced chemical modifications selected from the group consisting of lipid oxidation, protein crosslinking, amino acid oxidation and amino acid modification.

48-57. (canceled)

58. The enveloped virus based virus like particle preparation of claim 44 further comprising a hemagglutinin polypeptide, a respiratory syncytial virus M polypeptide, a respiratory syncytial virus G polypeptide, a respiratory syncytial virus F polypeptide, or combinations thereof.

59-61. (canceled)

62. The enveloped virus based virus like particle preparation of any of claim 44, wherein the enveloped virus-based virus-like particles comprise:
(a) a gag polypeptide; and
(b) an hemagglutinin polypeptide.

63. The enveloped virus based virus like particle preparation of claim 44, wherein the enveloped virus-based virus-like particles comprise:
(a) an influenza M1 polypeptide; and
(b) an hemagglutinin polypeptide.

64-70. (canceled)

71. A method for treating or preventing a disease or symptom of the immune system, comprising administering an immunogenic amount of the enveloped virus based virus like particle preparation of claim 44.

72. The method of claim 71, wherein the administering induces a protective immunization response in the subject.

73. The method of claim 71, wherein the administering is selected from the group consisting of subcutaneous delivery, intradermal delivery, subdermal delivery, intramuscularly delivery, peroral delivery, oral delivery, intranasal delivery, buccal delivery, sublingual delivery, intraperitoneal delivery, intravaginal delivery, anal delivery and intracranial delivery.

74. A pharmaceutical composition comprising an immunogenic amount of the enveloped virus based virus like particle preparation of claim 44.
